(19) United States
(12) Patent Application Publication
(10) Pub. No.: US 2024/0076738 A1
(43) Pub. Date: Mar. 7, 2024

(54) Methods for Measuring Analytes in Cell Subpopulations

(71) Applicant: Cleveland Cytometry Services Company, Novelty, OH (US)

(72) Inventors: James W. Jacobberger, Novelty, OH (US); Philip G. Woost, North Ridgeville, OH (US)

(21) Appl. No.: 18/241,855

(22) Filed: Sep. 2, 2023

Related U.S. Application Data
(60) Provisional application No. 63/403,812, filed on Sep. 5, 2022.

(51) Int. Cl. C12Q 1/6881

(52) U.S. Cl. CPC C12Q 1/6881

(57) ABSTRACT

Disclosed are methods for distinguishing subpopulations of cells within a cell population and determining analyte expression in the subpopulations. Herein, subpopulations of cycling and non-cycling cells were identified in a cell population based on expression of a first analyte (e.g., Ki-67). Levels of a second analyte were determined in the cycling cells, exclusive of the non-cycling cells.

### CROSS-REFERENCE TO RELATED APPLICATION

This application claims priority to U.S. Provisional Patent Application Ser. No. 63/403,812, filed Sep. 5, 2022.

### BACKGROUND

Detecting, measuring, and quantifying analytes (e.g., products of gene expression or metabolic processes) in biological cells is used to understand cell biology. These methods are also used in diagnostics, where detection of aberrant expression of a cellular analyte in cells from a patient or healthy individual may indicate disease or presence of a progressive disease producing process. The methods may also be used to determine efficacy of a therapeutic agent in modifying levels of cellular analytes that are surrogate markers for or direct contributors to disease or disease producing processes. These methods are also used in drug discovery, where therapeutic drug candidates are screened for their ability to alter expression of specific cellular analytes that are direct targets or markers for upstream targets.

In some instances, it may be desirable to detect, measure and/or quantify cellular analytes in subpopulations of cells in a cell sample. For example, as in rare cells that express an analyte but exist within a large population of cells that do not express the analyte, but increase the difficulty of detecting, measuring, and quantifying the analyte. For example, it may be of interest to detect/measure/quantify analytes in cycling cells versus non-cycling cells, or in cells in individual cell-cycle compartments (e.g., G1, S, G2, M, G0), or in cells of specific lineages or differentiation/maturation states. In some instances, these measurements may be obtained from individual cells (i.e., on a single-cell basis), rather than as an average measurement of all cells in a population (e.g., as might be obtained for an epitope or epitopes using a Western blot, or peptides or residues by mass spectrometry).

There is interest in developing methods that better and more accurately detect, measure and/or quantify analytes in subpopulations of biological cells.

### SUMMARY

Disclosed here are methods for determining amounts of analytes in subpopulations of biological cells. The methods are designed to distinguish subpopulations of cells within a population of biological cells, and to determine/identify levels of analytes in cells of the subpopulations and/or to determine and correlate other characteristics of the cells in the subpopulations. The methods are designed to exclude contribution of analytes from subpopulations other than the desired subpopulation(s) to the determination of analytes in the desired subpopulation. The methods for determining and/or identifying levels of analytes are generally performed on a single-cell basis—that is, the most basic measurement is made on individual cells or sub-cellular organelles.

In some examples, a first analyte is determined in a population of cells and at least one subpopulation of cells is identified, based on detection of the first analyte. One or more characteristics of the identified subpopulation of cells is identified. In some examples, a second or larger number of analyte(s) may be determined in the cells that make up the subpopulation. In some examples, cell state markers identify specific biochemical states of the target cell population(s) (e.g., based on the cell cycle, differentiation, maturation, or apoptotic state) and parameters may be determined for the cells of the subpopulation(s).

In some examples, cycling cells are distinguished from non-cycling cells, based on expression of a first analyte (e.g., a state marker), and second analytes are detected and/or quantified in the cycling cells or in the non-cycling cells. In some examples, cells in one state (phase) of the cell cycle (e.g., G1 phase) are distinguished from cells in other phases of the cell cycle, and analytes are detected and/or quantified in cells in one or more phase(s) of the cell cycle.

In some examples, in a population of biological cells containing cycling and non-cycling cells, cells in the G1-phase of the cell cycle are distinguished from non-cycling cells, referred to as in the G0 “phase”, meaning cells that are not in the cell cycle. G0 and G1 cells are both distinguished from other cell cycle phases by a structural or foundation state—that of having a quantity of DNA that codes for two genomes (i.e., referred to as 2N for genomes that encode a wild-type complement of DNA sequences for the organism; 2C refers to a stable amount of DNA that codes for aberrant genomes usually correlated with disease or propensity for disease). The G1-phase cells may be distinguished from the G0-phase cells, even though both G1- and G0-phase cells have a 2N or 2C DNA content, based on identification in the cells of an analyte that is differentially present or expressed in G1-phase cells as compared to G0-phase cells. A second analyte may then be determined/levels identified in the G1-phase cells, excluding the G0-phase cells, or may be determined/levels identified in the G0-phase cells, excluding the G1-phase cells. This avoids the situation where G1-phase cells are not distinguished from G0-phase cells, and levels of the second analyte in the combination of G1- and G0-phase cells is used as a proxy for levels of the second analyte in the G1-phase cells.

In some examples, the population of cells that is analyzed as above has been treated with or exposed to a candidate therapeutic agent. For example, T cells are identified using a set of first analytes (e.g., Type I analytes, phenotypic markers); G1-phase cells are identified using detection of one or more second analytes (e.g., Type II analytes; biochemical state markers), and a third analyte (e.g., Type III analytes; target analyte) is detected or quantified in G1-phase, T cells. Generally, the therapeutic agent does not affect expression of the Type I and II analytes. Use of cells treated with/exposed to candidate therapeutic agents may be used to test the ability of the candidate therapeutic agent to affect expression of the Type III analyte(s) in cycling cells when the Type III analyte is the target or a surrogate downstream “read out” target of the therapeutic agent. Use of cells treated with/exposed to candidate or approved therapeutic agents may be used to monitor patient or study subject responses to approved therapeutic agent(s) by controlling or modifying the expression of the Type III analyte(s) in cycling cells when the Type III analyte is the target or a surrogate downstream “read out” target of the therapeutic agent.

In some examples, the population of analyzed cells may be treated with combinations of therapeutic agents that include multiple therapeutics that target multiple Type III analytes or upstream regulators of Type III analytes, and the methods may include additional related series of Type II and Type III analytes to query the independent and interaction effects of multiple therapeutics in combination.

Generally, the methods include a concept of and explicit execution of sample quality assessment that provides an assessment of data quality. This feature may eventually lead to a quantitative evaluation of data quality. Over time, a large library of data quality assessments should provide quality assurance for future studies and improve the ability to be more precise when stating therapeutic potential of candidate therapeutics. Sample quality assessment may include several parametric indices (e.g., subpopulation ratios; distribution of target analyte levels at baseline in a patient or healthy population; aberrant expression of cell biochemical state markers; degraded morphology (assessed with light scatter), DNA/RNA/protein/lipid content; expression of cell stress and cell death markers)).

### BRIEF DESCRIPTION OF THE DRAWINGS

In the accompanying drawings, which are incorporated in and constitute a part of the specification, embodiments of the disclosed inventions are illustrated. It will be appreciated that the embodiments illustrated in the drawings are shown for purposes of illustration and not for limitation. It will be appreciated that changes, modifications, and deviations from the embodiments illustrated in the drawings may be made without departing from the spirit and scope of the invention, as disclosed below.

FIGS. 1A, 1B and 1C illustrate example data from studies of cell cycle distributions of DNMT1 in stimulated CD3⁺ T cells.

FIGS. 2A, 2B, 2C, 2D, 2E, 2F, 2G, 2H, 2I, 2J and 2K illustrate example analytical Boolean logic and sequential bivariate region setting to reduce the final sub-population for informative DNMT1 determinations.

FIG. 3 illustrates example data from studies of DNMT1 expression analysis that demonstrate the distribution of values for a human population and that the expected elevation of DNMT1 in S phase is measurable with high significance.

FIG. 4 illustrates example data from a study of healthy human volunteers before and after drug treatment demonstrating quantification of DNMT1 and the assay sensitivity to determine the effects of a drug.

FIG. 5 illustrates the power of single cell measurements and using Boolean logic to focus the measurements on cells that are most informative. The y-axis in the plot (DNMT1 Value) includes DNMT1+background fluorescence. The example results for median DNMT1 expression in cycling single cells in the G1-phase of the cell cycle (G1 cells) are compared to median DNMT1 expression in cycling single cells plus non-cycling single cells in all cell cycle phases (All Cells)—i.e., all cells were queried for DNMT1 expression and the background from the very large number of non-expressing cells overwhelms the measurement of the rare positive cells. The “All Cells” values are equivalent to results that would be obtained with a bulk assay like Western blots.

FIG. 6 illustrates example results for median DNTM1 expression levels in single cells in various cell cycle phases, for cells from untreated subjects (indicated as Baseline) and cells from decitabine-treated subjects (indicated as Treated). The y-axis in the plot (DNMT1) includes DNMT1+background fluorescence. For the most part, the values in G0 cells consist of background fluorescence and is not specific for DNMT1. This is normally subtracted from, for example, Late G1, to obtain data sets as presented in FIGS. 3 and 4.

FIG. 7 illustrates an example schematic diagram of the methods disclosed herein. Sampling a tissue (1^{st} row), detecting a tissue cell sub-population (2^{nd} row), and detecting cell processes (3^{rd} row) are shown. The diagram illustrates using cell state (defined as a stable period of time during which biochemical analytes are going up, are at steady state, or are going down in combination), coordinated by the overarching cell process, made possible by single cell measurements. Also shown is combining the three stages above with an endpoint that is a target of a drug or a target of a target of a drug. Rows 1-4 are represented with real data in FIGS. 2a-2c (row 1), FIG. 2d (row 2), FIGS. 2g-2h (row 3), and FIGS. 2I-2K (row 4). FIGS. 3 and 4 represent practical use of the methods.

### DETAILED DESCRIPTION

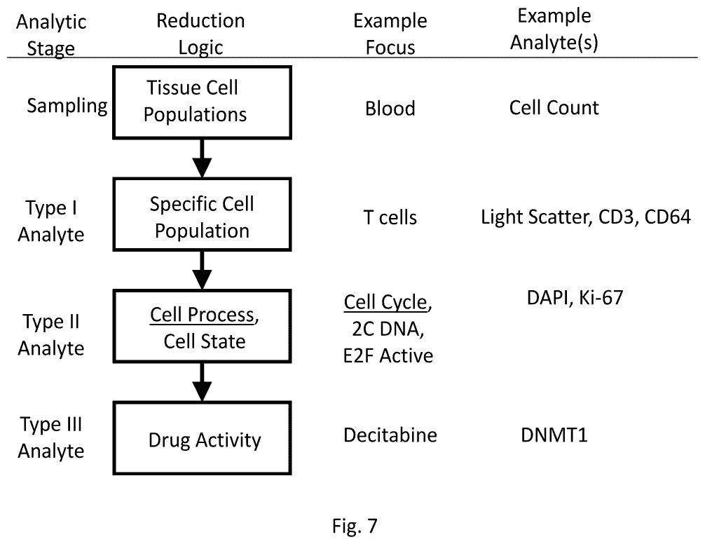

In molecular biology, measurements of cellular analytes (e.g., gene products) in cells commonly involves determining a mean level of an analyte for an entire population of cells. For example, a protein extract may be made from a dish of cultured cells, the proteins in the extract may be separated using SDS-polyacrylamide electrophoresis, and an analyte protein may be detected with an antibody as a band on a Western blot. Generally, the intensity of the band is proportional to the average amount of that analyte in the cell population. But, if the starting cell population contained multiple cell subpopulations, differences in levels of the analyte in the different subpopulations would not be detected in the Western blot. This disclosure describes rule-based methods for identifying certain subpopulations within a population of cells and determining levels of an analyte of choice (e.g., a specific Type III analyte) in the subpopulations, without contribution from cells that are not part of the subpopulations. The methods are further reduced by Boolean isolation to specified cell states based on dominant transcription factors, active enzymes, signaling activity, etc. (e.g., cell cycle phases, sub-phases, mitotic compartments, DNA damage responses, DNA repair processes, cell death processes, etc.). The complexity of analysis to determine the cell state level can be found in this publication (Jacobberger J. W., Sramkoski R, M., Stefan T., Woost P. G. (2018) Multiparameter Cell Cycle Analysis. In: Hawley T., Hawley R. (eds) Flow Cytometry Protocols. Methods in Molecular Biology, vol 1678. Humana Press, New York, NY. doi.org/10.1007/978-1-4939-7346-0_11). A schematic of the disclosed methods is shown in FIG. 7.

In some examples, a cell sample (e.g., PBMCs from a healthy donor, patient, or research animal), may contain both cycling and non-cycling cell subpopulations. In this cell sample, if there is interest in determining levels of an analyte only in the cycling cell subpopulation, then there should be a way to exclude non-cycling cells from the analysis. Conversely, in such samples, if there is interest in determining levels of an analyte only in the non-cycling cell subpopulation, then there should be a way to exclude cycling cells from the analysis. If there is interest in determining analyte levels in cells in individual phases of the cell cycle (e.g., G1, S, G2), there should be ways to distinguish cells in the desired cell cycle phase from cells in other phases of the cell cycle. This disclosure describes such methods.

Cells in certain phases of the cell cycle can already be crudely distinguished from one another based on DNA content. Non-cycling (G0) and cells in the G1 cell cycle phase have a 2N or 2C DNA content. Cells in S-phase of the cell cycle have between 2N/C and 4N/C DNA content. Cells in the G2 and M cell cycle phases have a 4N/C DNA content (M phase includes all the mitotic stages). However, cells in some cell cycle phases cannot be distinguished from one another based on DNA content. For example, both G1-phase (cycling cells) and G0-phase (non-cycling cells) have a 2N/C DNA content. Therefore, in a population of cells having cycling and non-cycling cells, the subpopulation of cells that has a 2N/C DNA content will contain both G1-phase and G0-phase cells. Expression of certain genes will be different in these two cell subpopulations and, therefore, it may be desirable to determine expression of those genes in G1- or G0-phase cells separately. The same is true for 4N/C cells. G2 phase and M phase cells are biochemically very different cell types and measuring gene expression (RNA, protein, protein modifications) is highly regulated and variably expressed in restricted cell states within these broad phases.

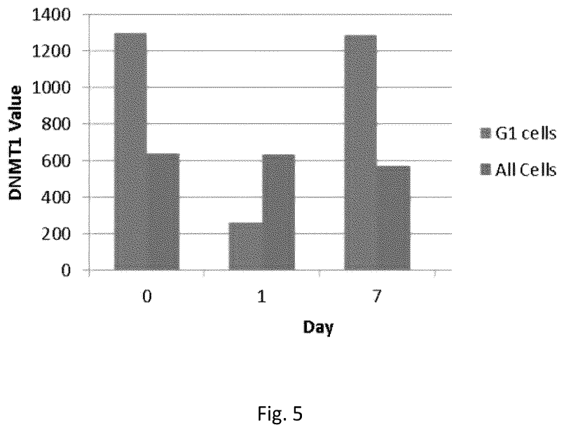

Consider an example where there is interest to measure levels of a Type III analyte in the cycling cells in a cell sample. This analyte is known to be expressed in the G1-phase of the cell cycle, but not to be expressed in the G0-phase of the cell cycle. Consider also that the cell sample used for the analysis is a sample of peripheral blood mononuclear cells (PBMCs), which generally contain about 5% or less cycling cells. If the PBMCs are stained for DNA (e.g., using DAPI), stained for the Type III analyte (e.g., using a specific antibody), the stained cells analyzed by flow cytometry, and the levels of the Type III analyte determined in cells having a 2N/C DNA content, the determined analyte level would underestimate analyte levels of G1-phase cells. In other words, levels of the first analyte in cells having a 2N/C DNA content is not a substitute for measuring levels of the first analyte in G1-phase cells. This is demonstrated in FIG. 5 where the Type III analyte level measurement is almost entirely diminished by background noise (All cells). Whereas, by the single-cell method herein (G1 cells), the noise is removed and the baseline signal (day 0) is ˜6× the treated signal (day 1), a pattern which is repeated at 7 days (recovered, untreated) and day 22 (treated).

In some examples, herein, methods are disclosed for measuring expression of Type III analytes in cycling cells (or in non-cycling cells) that are present in a cell population containing both cycling and non-cycling cells.

Consider the above example, in which PBMCs were stained for DNA and a Type III analyte. Now, consider that the cells were also stained with an antibody specific for an additional analyte (e.g., Type I or II) known to be differentially expressed in cycling (e.g., G1) versus non-cycling (e.g., G0) cells. When the stained cells are analyzed by flow cytometry, cells with a 2N/C DNA content can be identified as cycling (G1-phase cells) or non-cycling (G0-phase cells), based on differential expression of the additional, Type I/II analyte. Levels of the Type III analyte in the cycling, G1-phase cells can be determined independently of the G0-phase cells. Any “dilution” in levels of the Type III analyte, obtained when all cells having 2N/C DNA content were grouped, is avoided.

In some examples, therefore, the methods disclosed herein are useful for distinguishing different subpopulations of biological cells based on cell state, that have the same DNA content, from one another, and determining Type III analytes in the distinct subpopulations (cell states), separate from other subpopulations (cell states). The disclosed methods exemplify distinguishing G1-phase cells from G0-phase cells (both having 2N/C DNA content) and determining levels of one or more analytes (e.g., DNA methyltransferase 1) in the G1-phase cells.

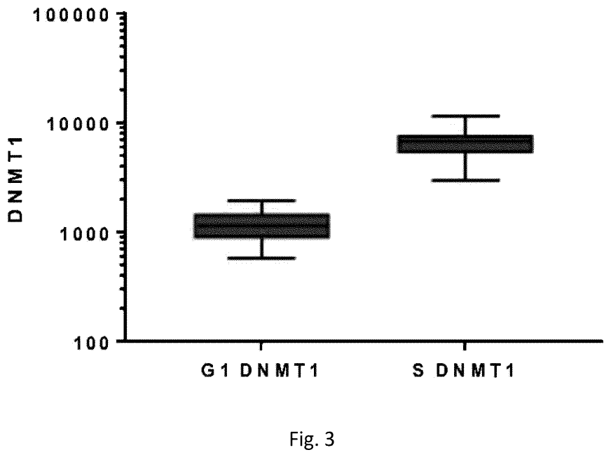

Included in the G1 phase analysis described by the preceding sections, the method also produces S phase measurements. For the example presented, the Type III analyte, DNMT1, is normally expressed at a well-regulated higher level in S phase cells (FIG. 3). This produces a sample level quality assurance in sample from untreated, control patients, or from baseline measurements for treated patients.

In some examples, the cell population to be analyzed, having both cycling and non-cycling cells, may have been treated with a candidate or approved therapeutic agent. In examples where the candidate or approved therapeutic agent affects expression of a Type Ill analyte in the cells, the methods disclosed here can be used to identify, in G1-phase or other cell-state subpopulations of cells, changes in analyte expression as a result of the candidate or approved therapeutic agent. This is illustrated in FIGS. 3, 4, 5, and 6.

Generally, the methods described here provide a way to use a sequential restricting analysis of the cell population in a patient, donor, or animal tissue sample that contains only a small number of a cycling subpopulation cells (e.g., G1 T cells), to quantify an important endpoint (Type III analyte) that responds to candidate or approved therapeutic agents, recovery from the candidate therapeutic or approved agents, and re-treatment with candidate or approved therapeutic agents.

Unless defined otherwise, all technical and scientific terms used herein, with the exception of the designation of analytes as Type I, Type II, and Type III, have the same meaning as commonly understood by one of ordinary skill in the art to which the present invention pertains. It is to be understood that the terminology used herein is for describing particular embodiments only and is not intended to be limiting. For purposes of interpreting this disclosure, the following description of terms will apply and, where appropriate, a term used in the singular form will also include the plural form and vice versa.

Herein, “analyte” refers to a substance that is being identified and/or measured. Generally, this application refers to analytes from and/or produced by cells (i.e., cellular analytes). Any or all substances (e.g., molecules) from or produced by a cell may be referred to herein as cellular analytes. Chemically, cellular analytes may include proteins, polypeptides, peptides, saccharides, polysaccharides, lipids, nucleic acids, other biomolecules, and modified residues on macro molecules (e.g., phosphorylation, methylation, etc.).

In some examples, three types of analytes may be considered. A first (type I) includes phenotype markers. These may be used, but not exclusively, to define cell populations and subpopulations based on differentiation and maturation states. Examples of type I analytes include CD45, CD3, CD4, CD5, etc.

A second type of analyte includes cell biochemical state markers. These may be used to reduce a phenotypically defined cell subpopulation to cell process-based compartments with defined baseline levels of a third type of analyte that may constitute target analyte(s) of a study and satisfy a rationale for the assay. For example, sets of phenotype analytes may define subsets of T cells circulating in peripheral blood. Cell cycle analytes may define non-cycling and cycling cells and further reduce the subpopulation of cycling cells to specific subtypes of cycling cells (G1, S, and G2 phases, mitotic cells etc.). Examples of type II analytes include (cell cycle markers) Ki-67, PCNA, RB1, and other E2F-activated proteins, phosphorylated epitopes on E2F activated proteins, subtypes of cyclins A, B, D, E, and epitopes that read out activity of cell cycle-regulating specific kinases.

A third type of analyte may inform a diagnosis, study, and/or clinical trial, generally indicating but not limited to a patient diagnosis, change in diagnosis, patient monitoring, or patient response to therapy. Examples of type III analytes include the family of enzymes that respond to differentiation therapeutics (azacytidine, decitabine)—DNMT1, UCK2, DCK, CDA. In this case, the primary target is DNMT1, which is the protein that is depleted by hypomethylating drugs, CDA which inactivates the drugs, UCK2 and DCK that switch dominance in response to hypomethylating drugs and provide an alternate pathway for cells to escape the action of the drugs.

Herein, “analyze” refers to investigating or quantifying by established cytometric methods. Herein, biological cells or cell organelles are generally analyzed for analytes by cytometric methods.

Herein, “antibody” or “antibody binding agent” generally refers to a molecule or molecules (e.g., protein) that binds an antigen. Herein, “antibody” generally refers to all types of antibodies, fragments and/or derivatives. Antibodies include polyclonal and monoclonal antibodies of any suitable isotype or isotype subclass. Herein, antibody may refer to, but not be limited to Fab, F(ab′)₂, Fab′ single chain antibody, Fv, single chain, mono-specific antibody, bi-specific antibody, tri-specific antibody, multi-valent antibody, chimeric antibody, canine-human chimeric antibody, chimeric antibody, humanized antibody, human antibody, CDR-grafted antibody, shark antibody, nanobody (e.g., antibody consisting of a single monomeric variable domain), camelid antibody (e.g., from the Camelidae family) microbody, intrabody (e.g., intracellular antibody), and/or de-fucosylated antibody and/or derivative thereof. Mimetics of antibodies are also provided.

Herein, “assay” refers to a method for analyzing.

Herein, “bind” refers to contacting and securing; forming a complex with.

Herein, “binding agent” includes substances that can specifically bind to other substances. Binding agents may include proteins, peptides, haptens, nucleic acids, etc.

Binding agents include antibody binding agents.

Herein, “biological” refers to life or living.

Herein, “biological cell” refers to the basic membrane-based unit that contains the biomolecules of which living things are composed (i.e., a discrete biological system). Some cells may themselves be organisms (e.g., prokaryotic cells are examples of this). Some cells (e.g., some eukaryotic cells) may be part of organisms or tissues.

Herein, “blood” refers to the fluid from an organism that carries oxygen and nutrients to cells of the organism and carries carbon dioxide and other waste products from cells of the organism.

Herein, “capable” refers to having the ability or quality to do something.

Herein, “cell cycle” refers to the phases a cell progresses through as it undergoes cell division and the cell population proliferates. Generally, cells progress sequentially through G1 (Gap 1), S (DNA synthesis), G2 (Gap 2) and M (mitosis) phases of the cell cycle. G1-phase cells are said to contain a 2N/C amount of DNA. S-phase cells contain between a 2N/C and 4N/C amount of DNA. G2-phase cells have a 4N/C amount of DNA. In M phase, cells have a 4N/C amount of DNA, which divides into two daughter nuclei. At the end of mitosis, the cell divides into two progeny cells each with a 2N/C amount of DNA. Cells progressing through these phases may be said to be “in” the cell cycle or may be referred to as “cycling” or “proliferating” cells. After mitosis, depending on environmental factors, progeny cells can either enter a new G1 phase, which will biochemically be identical to the previous G1 phase, or enter G0, which will be biochemically identical to all cells of that type residing in a G0 state. Based on content of DNA in single cells of a cycling cell population, cells may be classed as G0+G1-, S-, or G2+M-phase cells (i.e., M-phase cells have a 4N/C DNA content until the cells physically divide and M phase ends).

Cells not progressing through the cell cycle may be said to be “non-cycling” or “quiescent” cells. Non-cycling cells generally are not progressing or are not “preparing” to progress through the cell-cycle phases described above (G1, S, G2+M). Generally, herein, non-cycling cells are in a phase or state called G0. Like G1-phase cells, G0-phase cells have a 2N/C amount of DNA.

Herein, “cell cycle distribution” refers to percentages of the cells in a cell population of subpopulation that are present in individual phases of the cell cycle. For example, in a population of cycling cells, the cells may be distributed in the cell cycle as follows: 50% of the cells may be in the G1-phase of the cell cycle, 35% may be in the S-phase of the cell cycle and 15% of the cells may be in the G2- or M-phase of the cell cycle.

Herein, a “cell cycle phase marker” refers to a cellular analyte that is differentially present in cells of a particular cell cycle phase, as compared to another cell cycle phase. One example of a cell cycle phase marker is Ki-67. Ki-67 is expressed in G1, S, and G2+M-phase cells but not in G0-phase cells. Therefore, Ki-67 determination in a population of cells can be used to distinguish cells with a 2N/C DNA content that contain Ki-67 (G1-phase, cycling cells) from cells with a 2N/C DNA content that do not contain Ki-67 (G0-phase, non-cycling cells). Such analytes are referred to as proliferation markers because they are expressed in almost all cycling cells. Another such analyte is PCNA, which is also co-expressed like Ki-67. Both are E2F activated genes. E2F is required for cell cycle progression and activated at the transition from G0 to G1. Examples of cell cycle state specific markers are cyclins D, E, A, and B.

Herein, “chemical” refers to compounds, generally synthetically made.

Herein, “cycling”, in reference to cells, refers to cells that are in one of the G1, S, G2 or M cell cycle phases. Generally, cycling cells in one of these cell-cycle phases are progressing to the subsequent cell cycle phase (e.g., G1-phase cells are progressing to S phase; S-phase cells are progressing to G2 phase; G2-phase cells are progressing to M phase; M-phase cells are progressing to G1 phase).

Herein, “deoxyribonucleic acid” or “DNA” refers to a nucleic acid formed from polymerization of deoxyribonucleotides.

Herein, “determine” refers to detecting something and/or quantifying something.

Herein, “difference” refers to something that is not the same. “Differential” may refer to things that are not the same. In some examples, “differentially” may be used to refer to a cellular analyte that is present in one cell but not another. In some examples, “differentially” may be used to refer to a cellular analyte that is present in different amounts or levels in different cells.

Herein, “distinguish” refers to discerning one thing from another based on one or more characteristics of the things.

Herein, “DNA content” generally refers to the amount of DNA in a cell or a relative amount of DNA within a single cell as compared to other cells.

Herein, “DNA methyltransferase” or “DNMT” refers to enzymes that methylate cytosine in CpG dinucleotides. One example is DNMT1. DNMT1 is responsible for methylation of replicating DNA containing the CpG dinucleotides during S phase. Transcription of genes may be downregulated by methylation of the CpG dinucleotides. This is the basis of hypo-methylating therapy with azacytidine or decitabine. Genes that are normally repressed by methylation are activated with these drugs that directly target DNMT1. Some of these genes reprogram cancer cells to differentiate to a harmless, non-proliferating state for example.

Herein, “DNA methyltransferase inhibitor” or “DNMT inhibitors” refers to substances that can inhibit methylation activity of DNA methyltransferases. Transcription of normally or abnormally repressed genes may be upregulated by inhibition of DNMT activity. Example DNMT inhibitors include decitabine and azacytidine.

Herein, “dye” refers to a substance that is usually fluorescent and adds color or changes color of something. The color is not necessarily detected by the human eye (e.g., uv excited dyes).

Herein, “E2F” refers to a group of cellular transcription factors and/or refers to genes that encode E2F transcription factors. The various E2F transcription factors may generally be either target gene activators or target gene repressors. Herein, the E2F transcription factors that activate target genes (e.g., E2F1, E2F2, E2F3a) are of interest. These activators activate transcription of a group of genes that regulate transition of cells between the G1 and S phases of the cell cycle (i.e., the G1/S transition). Herein, the target genes activated by E2F activators may be called “E2F target genes.”

Herein, “effusion” refers to an abnormal escape or accumulation of a bodily fluid.

Herein, “exclusive of” refers to not including something. In reference to determining a cellular analyte in a first group or subpopulation of cells exclusive of a second group or subpopulation, this means that the value ascribed, for example, to the mean median level of the analyte in the first group of cells does not include any contribution from the analyte in the second group of cells.

Herein, “exposed to” generally is used in reference to cells that have been exposed to, for example, a candidate therapeutic agent. In some examples, the cells may have been exposed to a candidate therapeutic agent in vitro. In some examples, the cells may have come from a patient to whom a candidate, repurposed, or approved therapeutic agent has been administered.

Herein, “expression”, with respect to an analyte (e.g., mRNA, protein, or the like), refers to production and presence of an analyte in a cell.

Herein, “flow cytometry” generally refers to analysis obtained by flow of single cells through an energy source (e.g., laser).

Herein, “cytometry” includes flow cytometry as well as laser scanning cytometry or any other microscopic technology that facilitates making measurements on single cells.

For example, mass-spectrometer based cytometers.

Herein, “fluorescent” generally refers to a property of some substances (including dyes) to absorb energy at one wavelength and to emit energy at another wavelength.

Detection of the emitted wavelength is a basis for some assays used herein.

Herein, “independent of” refers to excluding something.

Herein, “identify” refers to ascertaining or establishing something.

Herein, “imaging” refers to producing a visual representation of something.

Herein, “level”, generally in reference to an analyte, refers to the amount of the analyte in or associated with a cell or cells.

Herein, “mononuclear cells” refers to cells from blood that have a single, ˜round nucleus (not lobulated). Mononuclear cells from blood may be called peripheral blood mononuclear cells (PBMCs). Physical preparation of PBMC samples eliminate granulocytes and red blood cells.

Herein, “non-cycling” in reference to cells, refers to cells that are in the G0-phase of the cell cycle. Cells may reside in the G0 phase indefinitely. Cells in the G0 phase are not progressing to S phase.

Herein, “population”, in reference to cells, generally refers to multiple cells.

Herein, “provide” refers to supplying or making something available.

Herein, “quantify” refers to ascertaining an amount or relative amount, generally of an analyte.

Herein, “reagent” refers to a substance or mixture of substances for use in analyzing and/or in an assay.

Herein, “sample” generally refers to a collection of something. In some examples, “sample” may refer to a collection of biological particles (e.g., cells). In some examples, a biological sample may comprise any number of macromolecules, for example, cellular macromolecules. The sample may be a cell sample. The sample may be a cell line or cell culture sample. The sample can include one or more cell types or cells, or one or more cell aggregates or clusters, composed of a uniform or heterogenous cell type composition. The biological sample may be derived from another sample. The sample may be a tissue sample, such as a biopsy, core biopsy, needle aspirate, or fine needle aspirate. The sample may be a fluid sample, such as a blood sample, pleural effusion, ascites, cerebral spinal fluid, urine sample, or saliva sample. The sample may be a skin or colon biopsy sample. The sample may be a cheek swab. The sample may be a plasma or serum sample. The sample may be a blood or bone marrow sample. The sample may contain PBMCs.

Herein, “single cell” generally refers to a cell that is not present in an aggregate or clump.

Herein, “single cell-basis” is generally used in the context of describing something (e.g., an analyte) within or associated with an individual cell. An analyte measured on a single-cell basis is generally measured in individual cells. For example, the amount of an analyte in an individual cell may be determined for each cell in a population of cells (i.e., using cytometry). The single-cell measurements may be expressed, for example, as a distribution of the individual cell determinations. A central value of the of the distribution (mean, percentiles, median, mode) may be and is usually determined and reported. This contrasts, for example, with determination of an average level of an analyte for example, in a population of cells that does not involve single-cell measurements (e.g., a Western blot of a cellular extract obtained from a population of cells).

Herein, “single-cell method” refers to methods used to analyze cells on a single-cell basis. Single-cell methods may include flow cytometry, microscopic imaging, single-cell sequencing, and the like.

Herein, “single-cell sequencing” refers to nucleotide sequencing methods whereby the obtained sequences can be ascribed to individual single cells.

Herein, “stain” refers to contacting cells and/or a cellular substance or organelle with a substance capable of binding to the cellular substance or organelle. Herein, DNA in a cell may be stained with, for example, propidium iodide, DAPI, and other agents. Herein, analytes in a cell may be stained with, for example, an antibody.

Herein, “subpopulation” in reference to cells, refers to cells within a population that are grouped together based on one or more characteristics. A subpopulation of cells is generally less than or a part of a population of cells.

Herein, “substance” refers to a physical material.

Herein, “with no addition from” generally refers to levels of an analyte in a cell subpopulation that is independent of the analyte in another cell subpopulation.

Herein, “therapeutic agent” generally refers to substances that treat or ameliorate a condition in a patient. Therapeutic agents whose effects are known may be referred to as established or approved therapeutic agents. DNA methyltransferase inhibitors, like decitabine and azacitidine, are established therapeutic agents that are approved for some diseases (myelodysplastic syndrome (MDS), acute myeloid leukemia (AML) and under current investigation for others (e.g., sickle cell anemia, AML suppression post bone marrow transplant). Therapeutic agents whose effects may not be know can be referred to as candidate therapeutic agents. The word “candidate” generally indicates a substance whose effect on gene expression or cell process(s) may be known or unknown. The methods described herein may be used to test candidate therapeutic agents for effects on gene expression, cell processes, or other parameters (e.g., catastrophic events like cell lysis).

### Types of Analytes

Generally, the methods disclosed herein are designed to identify a functionally homogenous cell subpopulation within a broader, heterogeneous cell population, identify a subpopulation of a subpopulation, and so on, until one can measure a parameter with increased precision of the identified subpopulation or sub-, subpopulation that is uniform.

Table 1 lists example categories of analytes used to identify sub- and sub-subpopulations. This reduction can minimize biological variation and improve the quality of a cytometric pharmacodynamic assay.

Type I analytes generally refer to cell lineage or type analyte sets that define broad classes (Type 1a) and functional sub-sets (Type 1b) of cells. An example is CD3 to identify immune T cells and CD4 to identify “helper” T cells. These cells can be further reduced by adding addition Type 1b analyte binding reagents. The choice of CD3 and CD4 reduces the number of cells analyzed in a sample of human peripheral blood to 4-20% of leukocytes. It also selects a population that is more uniform in cell properties, and thus, measurements of biomolecules will be more uniform.

Type II analytes generally reduce a Type I population further by marking and permitting selection of cells undergoing a functional cell process. For example, the E2F activated Type II analytes, Ki67 and/or PCNA, identify cells that are replicating (doubling of cell mass and eventual division into 2 cells). These analytes can negatively mark non-replicating cells and allow the focus of the study to be on resting or G0 cells. Either selection further reduces the complexity of and increases the uniformity of the cell population that will be measured. Replicating CD4 positive T cells are less than 0.1-1% of leukocytes. Adding additional Type II analytes can serially further segment the marked process. In the example case, adding DNA content permits selection of cells in the G1 or S phases of the cell cycle, and thereby selecting a more uniform population of cells, and further reducing variability.

Type III analytes are the response analyte that can inform the investigator about the magnitude of a therapeutic effect. For example, DNMT is an E2F activated, cell cycle gene, that is depleted by the drugs azacitidine and decitabine. Adding this to the above example provides a structured cytometric pharmacodynamic assay for the activity of these drugs in patients undergoing therapy.

TABLE 1
Types of Analytes

| Analyte | | |
|---|---|---|
| Type | Description | Purpose |
| Ia | Broad differentiation-related | Sector a cell population based on broad |
| | population identifier | cellular function. |
| Ib | Maturation-related population | Sector a cell population based on narrow |
| | identifier | cellular function. |
| II | Biochemical or “pathway” | ¹Sector a specific cell by a dominant cell |
| | process defining identifier | process at the time of sampling. For |
| | | example, cell cycle, apoptosis, necrosis, |
| | | DNA damage, DNA repair, activation (ligand |
| | | signaling) are all programmed processes |
| | | that define a major behavior of a cell. These |
| | | are generally common to all cells. |
| III | Target analyte | This is the objective of a pharmacodynamic |
| | | assay. Generally, it is gene product or |
| | | modification of a gene product, measured |
| | | quantitatively, that will respond to a drug. |
| | | Therefore, it is either the drug target per se, |
| | | or it is a downstream target that will |
| | | decrease or increase in quantity due to the |
| | | action of the drug directly or on an upstream |
| | | target that regulates the analyte. |

¹These processes can be subdivided and cells classified within a biochemical state. In brief, this is the conjunction of several gene products or modifications of gene products or the levels of each that are stable for a long enough period so as to render a cell uniquely identifiable. For example, peak levels of cyclin A2, cyclin B1, and phospho-S10-histone H3, identify cells as in the mitotic state “P2” (1), which correlates with the morphologic state, prophase. Similar states can be defined for any pathway or process.

Cells, Cell Populations and Treatment of Cells with Therapeutic Agents

The methods disclosed herein include identifying subpopulations of cells within a heterogeneous biological cell population cells or tissues and determining characteristics of the cells in the subpopulation(s), without contamination from cells of other subpopulations that may exist within the population. The subpopulations identified can be any subpopulation of cells that has a unique analyte composition (e.g., subpopulations that can be defined or identified based on a set of Type I and Type II analyte expression patterns) with respect to another subpopulation. Solid tissue samples (e.g., biopsies of tumors, liver, kidney, etc.) can be fixed and sectioned then measured by technologies such as laser scanning cytometry or digested to single cell suspensions then measured by flow cytometry. Fluid tissues (blood, lymph, cerebrospinal fluid), exudates (e.g., ascites, pleural effusions, urine) or semi-fluid/solid tissues (e.g., bone marrow) can be digested to single cell suspensions and analyzed by flow cytometry. The analytical objective is to reduce the population to a subpopulation and a cell state within that subpopulation that will inform studies of therapeutic agent (e.g., drug) action on the cells of a healthy or diseased individual, human or animal.

In some examples, the methods disclosed here involve identifying cycling and/or non-cycling subpopulations of cells within a larger cell population or sub-population, based on differential presence or expression of one or more Type II analytes (e.g., a first analyte) in the cycling subpopulation relative to the non-cycling subpopulation, or in the non-cycling population relative to the cycling subpopulation. Further, the methods disclosed here involve determining one or more additional Type III analytes (e.g., a second analyte) within one or the other of the cycling and non-cycling subpopulations, without contamination of the analyte determination in the cycling or non-cycling subpopulations from other subpopulations.

Generally, the populations of biological cells used in these methods can be any type of cells. The cells may be eukaryotic, prokaryotic or archaean. The cells may be mammalian or non-mammalian cells. The cells generally may be from any species, including human. In some examples, the cells are from blood, umbilical cord, spleen, bone marrow and the like. The cells may be peripheral blood mononuclear cells (PBMCs). The biological cells may be cultured ex vivo, grown in vitro, or may be a cell sample obtained from a healthy donor or patient

In some examples, the populations of cells used in the methods may contain cycling cells or non-cycling cells. In some examples, the populations of cells used in the disclosed methods may contain both cycling and non-cycling cells. As known in the art, cycling cells generally progress sequentially through G1 (Gap 1), S (DNA synthesis), G2 (Gap 2) and M (Mitosis) phases of the cell cycle. G1-phase cells generally contain a 2N/C amount of DNA (i.e., diploid cells). S-phase cells contain between 2N/C and 4N/C amounts of DNA. G2-phase cells have a 4N/C amount of DNA. In M phase, cells have a 4N/C amount of DNA. At the end of M phase, the parent cell divides and the resulting progeny cells each usually have a 2N/C amount of DNA. In some cases, in malignant cells, division may be unequal. Cells progressing through these phases may be said to be “in” the cell cycle or may be referred to as “cycling” or “proliferating” cells. Non-cycling cells generally are out of cycle or said to be in the G0-phase of the cell cycle. G0-phase cells have a 2N/C amount of DNA, like G1-phase cells. Cells may reside in the G0 phase indefinitely. Cells in the G0 phase are not progressing to S phase but may be capable of progressing to S-phase under certain conditions after stimulation and after first passing through G1. For example, there is some evidence that cells can exit the cell cycle in S, G2, or M phases to enter states referred to as S0, G20, or M0.

In some examples, the populations of cells used in the methods disclosed herein may have been treated or contacted with various substances. In some examples, the populations of cells may have been contacted with one or more candidate or approved therapeutic agents. In some examples, the candidate or approved therapeutic agent may affect expression of certain analytes in the cell subpopulation being examined. As will be discussed later, the methods disclosed herein may be used in drug discovery to screen various candidate therapeutic agents for those that have a desired effect on a population or subpopulation of biological cells or to screen approved therapeutic agents for re-purposing to treat conditions that were previously not included in the approval process. As will be discussed later, the methods disclosed herein may be used to determine efficacy of a candidate therapeutic agent or established or approved therapeutic agent that is being used to treat a patient. In these cases, the methods may be used to assist in setting patient or research organism dosing.

Many different substances may be used as candidate therapeutic agents in the methods described herein. In some examples, candidate therapeutic agents may affect analyte expression in non-cycling or cycling cells. In some examples, the candidate therapeutic agents may include DNA methyltransferase inhibitors. DNA methyltransferase inhibitors may include decitabine and azacitidine. In some cases, these agents may be combined with other agents that inhibit enzymes that degrade or inactivate the primary agent. An example of this is the cytidine deaminase inhibitor (CDA), tetrahydrouridine. This combination is currently being tested in clinical trials for sickle cell disease, non-small cell lung cancer, and esophageal cancer. It may be that CDA levels vary between men and women, between races or ethnic groups, as a function of age, with diet, as well as a natural distribution of levels within any one group. Therefore, intracellular levels of CDA are one of many planned future Type III analytes. Other enzymes that affect the bioavailability and half-life of decitabine or azacytidine are uridine cytosine kinase 2 (UCK2) and deoxy cytidine kinase (CDK). These are also examples of additional Type III analytes that will inform hypomethylating studies.

### Analytes and Detection of Cell Subpopulations (Reduction of a Cell Population to Uniformity)

Herein, the techniques used to identify subpopulations of cells within a larger population of biological cells generally involve identification of analytes (e.g., a first analyte; e.g., Type I)) that are differentially expressed between cell type subpopulations (e.g., differentiation or functional-based immunophenotypes). The methods also generally involve quantifying expression of another, second type of analyte (e.g., a second analyte; e.g., Type II) in or on cells or cell organelles of the identified Type I subpopulation that inform one or more biochemical processes (e.g., cell proliferation; cell nutritional, stress, and damage responses; cell death, etc.). The methods also generally involve quantifying expression of one or more third type, target analytes (Type III) that are the focus or foci of scientific investigations, biomedical research, clinical trials, or diagnostic, prognostic, or patient monitoring, including assistance with drug dose setting.

Herein, sometimes, detection of Type I analytes can be dispensed with, using other methods to purify a population to a necessary degree (the amount needed for informative results to be generated). For example, T cells might be prepared by immunomagnetic negative selection and then further processed with Type II and Ill analytes as describe above and below.

One element disclosed herein is that the target, Type III analytes are rendered detectable and quantifiable with precision and robustness that is defined by a quality assurance system. The Type I and Type II single cell measurements of cell populations produces a natural data state where time signatures (profiles) that underlie that biochemical heterogeneity in a sample can be used to reduce that heterogeneity to biochemical homogeneity sufficient for highly sensitive, highly precise measures of Type III, target analytes that meaningfully inform scientific investigations, biomedical research, clinical trials, or diagnostic, prognostic, or patient monitoring, including assistance with drug dose setting.

In some examples, the subpopulation of cells identified within a larger population of biological cells includes cycling cells. Generally, cycling cells are distinguished from non-cycling cells. To identify cycling cells, analytes that are differentially expressed in cycling versus non-cycling cells are detected. “Differentially expressed” in this context refers to detectable differences in the amount of an analyte in cycling cells as compared to non-cycling cells. In some examples, such an analyte may be present in cycling cells and not present in non-cycling cells. The analyte may not be present in cycling cells and be present in non-cycling cells. In some examples, the analyte may be present in both cycling and non-cycling cells, but present at different, detectable levels in one subpopulation versus the other. Generally, there is some difference in levels or amounts of the analyte in one subpopulation (e.g., cycling cells) compared to other subpopulations (e.g., non-cycling cells) in the larger population of biological cells (e.g., cycling cells can be distinguished from non-cycling cells based on the differential expression).

The logic applied to cell cycling, above, can be applied to other cell processes, including but not limited to cell nutritional, stress, damage response, and death pathways.

In some examples, the analyte used to distinguish cycling cells from non-cycling cells may be differentially expressed in cells having the same DNA content. For example, the analyte may be present in G1-phase cells but not present in G0-phase cells. The analyte may not be present in G1-phase cells but be present in G0-phase cells. The analyte may be present in both G1- and G0-phase cells, but present at different, detectable levels in one subpopulation versus the other. Equally, if an analyte is expressed at quantifiable differences in different phases of the cell cycle, e.g., G1, S, G2 or M, then the ratios between phases become elements of quality assurance in baseline and untreated samples. Once enough data is generated on treated samples, the relationship between dose, dose timing, and Type III analyte levels becomes a measure of quality assurance as well as defining a therapeutic response.

In some examples, the analytes used to distinguish cycling cells from non-cycling cells may be present in cycling, G1-phase cells and not present or present at low levels in non-cycling, G0-phase cells.

In some examples, the analytes used to distinguish cycling cells from non-cycling cells include analytes that are expressed in a cell-cycle dependent manner (i.e., expressed in some phases of the cell cycle but not in other phases). Such analytes may be referred to as cell cycle phase markers. In some examples, the analytes used to distinguish cycling cells from non-cycling cells may be analytes whose expression occurs in the G1-phase of the cell cycle. In some examples, these analytes may play a role in transitioning G1-phase cells into the S-phase of the cell cycle. In some examples, expression of these analytes may be activated by E2F transcription factors. In some examples, the E2F transcription factors may be E2F transcription factors that activate E2F responsive genes, rather than repress E2F responsive genes.

In some examples, the analytes used to distinguish cycling from non-cycling cells may include DNA polymerase, thymidine kinase, dihydrofolate reductase, cdc6, HsOrc1, MCM5 and the like. In some examples, the analyte used to distinguish cycling from non-cycling cells may include Ki-67 or PCNA.

In some cases, analytes like Ki-67, which is nucleolar-localized and produces a primary electronic signal in a flow cytometer that is a sharp, high peak, may benefit from primary signal processing. Therefore, detection may be improved by using peak (or signal) height or width rather than an integrated signal, or a combination of height/integrated or width/integrated.

Generally, expression of the Type I analytes used to distinguish cell subpopulations in the methods disclosed herein is not affected by a candidate therapeutic agent, if any, that might be used to treat the cell population or research subject or patients providing samples used in the methods. Generally, expression of the Type II analytes used to distinguish cycling cells from non-cycling cells, or used to distinguish other cell process cell states, is not affected by a candidate therapeutic agent, if any, that might be used to treat the cell populations or research subjects or patients providing samples used in the methods.

Once a cell subpopulation of interest is identified using a set of one or more first Type I analytes, a distinct set of one or more Type II analytes is identified within the subpopulation. Generally, the level of the second analyte in the cell subpopulation is determined. Generally, the second analyte can be any analyte in or on a cell that is distinct from the first analyte that is used to identify the cell states within cell processes also termed cell subpopulations but might be better termed as sub-sub-populations.

In some examples, the Type III analytes measured in the identified cell state subpopulation may be expressed in G1-phase cells. In some examples, the analyte measured in the identified cell subpopulation may be differentially expressed in G1-phase cells as compared to G0-phase cells. In some examples, the second analyte may include DNA methyltransferases. In some examples, the second analyte may include DNA methyltransferase 1 (DNMT1). In some examples, the expression of the analyte measured in the identified cell subpopulation may be affected by a candidate or approved therapeutic agent used to treat the cell population in cultured cells or in samples provided by research subjects or patients in studies using in the disclosed methods.

In some examples, the Type III analyte set can include analytes that affect the bioavailability or half-life of a candidate or approved therapeutic. Examples of these can include cytosine deaminase (CDA), uridine-cytosine kinase 2 (UCK2), deoxycytidine kinase (CDK). These enzymes are variable in the human population and inactivate decitabine or azacitidine. Current clinical trials are testing co-treatments with decitabine and tetrahydrouridine (THU) as a CDA inhibiter to increase bioavailability and half-life of decitabine using oral formulations. Measuring both DNMT1 and CDA in the same cells may inform clinical trials, research, and eventually patient monitoring for optimization of dosages by direct measurement rather than surrogates like body weight, gender, and age. Additionally, co-measurement of DCK and UCK2 may inform whether azacitidine or decitabine is the optimal drug that achieves the same result—depletion of DNMT1.

Other Type III sets may comprise the targets of co-administered investigational or approved therapeutics in studies of or treatment with multi-modal therapeutic formulations. Examples of these Type III sets include the kinases of current investigational or approved anti-cancer therapeutics. Examples are phosphorylated Akt in AML that may or may not respond to FLT-3 inhibitors.

In some cases, DNA damage responses may inform the ability to either reduce cytotoxicity in healthy cells or increase cytotoxicity in abnormal or cancer cells. The same condition of short existence times for cells expressing high levels of, for example, DNA damage means that they also will be rare cells. In this case, these Type II analytes can serve as Type III analytes. Examples of this type of mixed analyte are H2Ax and cPARP.

In some examples, the Type II analyte set may comprise critical disease co-variables that affect the first line standard of care therapy. For example, serial measurement of TP53, a stress response transcription factor, or downstream TP53-regulated gene products may alter therapeutic choices in MDS or AML, since TP53 mutant clones respond poorly to conventional chemotherapy. A cell death pathway Type II set could inform the efficacy of cell killing in for example, leukemias. The cells that express death pathway epitopes may exist for a short period of time and therefore may be rare. This invention should be able to determine significant changes in the cell death responses to investigational or approved therapies. Example Type II cell death analytes are BCL2 or cleaved caspace 3 among many others.

In the methods described herein, analytes can be detected/measured using a variety of techniques known in the art. Generally, the methods used include “single-cell methods” where analytes are interrogated on a per-cell basis. Single-cell methods may include techniques like single-cell sequencing of mRNA (i.e., transcriptomics), microscopic cell imaging, flow cytometry, acoustic focusing flow cytometry, laser scanning cytometry, and the like.

In some examples, flow cytometry is the single-cell method used to analyze cell populations in the methods disclosed herein. In these methods, the cell population to be used in the methods are generally fixed, permeabilized, or fixed and permeabilized using methods known in the art. The cells may then be “stained” to detect parameters of the cells, like presence of an analyte, an analyte set, multiple analyte sets and/or DNA, RNA, protein, lipid, or carbohydrate contents or modified residues. Examples of a modified residue are methyl or hydroxy-methyl cytosine residues in DNA.

Generally, staining to detect a cellular analyte may use an antibody or antibody binding-like agent that is specific for the cellular analyte. Generally, a labeled secondary antibody that binds to a first, primary antibody may also be used. The secondary antibody may be labeled with a fluorochrome or a primary antibody may be labeled with a fluorochrome. These techniques are known in the art. One example of these techniques is described in the following reference: Jacobberger, J. W., Fogleman, D. and Lehman, J. M., 1986. Analysis of intracellular antigens by flow cytometry. Cytometry: The Journal of the International Society for Analytical Cytology, 7(4), pp. 356-364.

In the methods described herein, the cell population may also be stained for DNA content. Generally, permeabilized cells or permeabilized cells that have been fixed are contacted with a substance that binds DNA. In some examples, the substance that binds DNA may be a dye. In some examples, the dye may be non-fluorescent or dimly fluorescent. In some examples, the dye may be a fluorescent dye. In some examples, the dye solution may include DAPI (4′,6-diamidino-2-phenylindole), propidium di-iodide, and the like. Dyes may also include ethidium bromide, Hoechst 33342, 33258, and other related dyes, SYBR and the like.

### Applications of the Methods

The methods disclosed here may be used to screen substances (e.g., candidate or approved therapeutic agents, lead compounds as a basis for therapeutic development, nutritional supplements, biochemical pathway inhibitors and agonists, and similar agents) for their effects on expression of certain analytes to monitor the effects of these agents on expression of certain analytes at one or more time intervals after treatment of research subjects (animals or humans), patients in clinical trials, or patients under a physician's care. The purposes are to assist in disease diagnosis or prognosis, monitor development of resistance to a therapeutic agent or agents, monitor development of clonal variants (e.g., a sub-population with the target subpopulation with altered analyte responses in the presence of a therapeutic or therapeutic agent(s).

In some cases, the methods disclosed here may be used to build a complex assay for a specific purpose (e.g., monitoring DNMT1 expression) and then combined with another complex assay for built for a different specific purpose (e.g., monitoring expression of an analyte, e.g., BCL2, in studies and clinical trials of multi-modal therapies). One purpose for combining assays is to maximize the value of patient's samples and to avoid unnecessary sampling.

In some cases, the methods disclosed here may be used with additional platforms to create integrated multi-platform assays that inform research studies and clinical trials. An example of the latter is to subject samples prepared by the methods disclosed here to fluorescence-based cell sorting for the purpose of isolating single cells of a cell sub-population (cell state) informed by the Type III analyte(s) that will be further interrogated by methods such as single cell sequencing of RNA or DNA.

### Embodiments

1. A method, comprising:
in a cell population that includes cycling cells and noncycling cells, determining a cellular analyte in the cycling cells exclusive of the non-cycling cells, or determining a cellular analyte in the non-cycling cells exclusive of the cycling cells.

2. The method of embodiment 1, wherein the cycling cells include cells in G1-phase of the cell cycle and the non-cycling cells include cells in G0-phase of the cell cycle.

3. The method of embodiment 1, wherein determining a cellular analyte includes quantifying a level of the cellular analyte.

4. The method of embodiment 1, wherein determining the cellular analyte uses single-cell methods.

5. A method, comprising:
in a population of cells containing cycling and noncycling cells, distinguishing the cycling cells from the non-cycling cells; and
determining a first cellular analyte in the cycling cells exclusive of the first cellular analyte in the non-cycling cells, and/or determining a first cellular analyte in the non-cycling cells exclusive of the first cellular analyte in the cycling cells.

6. The method of embodiment 5, wherein distinguishing the cycling cells from the non-cycling cells includes determining a second cellular analyte in the population of cells, wherein the second cellular analyte is differentially expressed in the cycling cells as compared to the non-cycling cells.

7. The method of embodiment 6, wherein determining the first cellular analyte and the second cellular analyte is performed on a single-cell basis.

8. The method of embodiment 5, wherein distinguishing the cycling cells from the non-cycling cells includes determining a second cellular analyte in the population of cells, wherein the second cellular analyte is differentially expressed in G1-phase cells as compared to G0-phase cells.

9. The method of embodiment 8, wherein the second cellular analyte includes a cell cycle phase marker.

10. The method of embodiment 8, wherein the second cellular analyte includes analytes whose expression is activated by E2F.

11. The method of embodiment 8, wherein the second cellular analyte is selected from the group consisting of DNA polymerase, thymidine kinase, dihydrofolate reductase, cdc6, HsOrc1, and MCM5.

12. The method of embodiment 8, wherein the second cellular analyte includes Ki-67.

13. The method of embodiment 5, wherein the first cellular analyte is differentially expressed in G1-phase cells as compared to G0-phase cells.

14. The method of embodiment 5, wherein the first cellular analyte includes a DNA methyltransferase (DNMT).

15. The method of embodiment 14, wherein the first cellular analyte includes DNMT1.

16. A method, comprising:
a) providing a population of cells that includes cycling cells and non-cycling cells;
b) distinguishing between the cycling cells and the non-cycling cells; and
c) identifying a first cellular analyte in the cycling cells or in the non-cycling cells.

17. The method of embodiment 16, wherein the first cellular analyte is identified in the cycling cells, with no addition from the non-cycling cells, or is identified in the non-cycling cells, with no addition from the cycling cells.

18. The method of embodiment 16, wherein the population of cells includes cells from blood, umbilical cord, spleen and/or bone marrow.

19. The method of embodiment 18, wherein the population of cells includes mononuclear cells.

20. The method of embodiment 16, wherein the cycling cells include at least some cells in G1-, S-, G2- or M-phases of the cell cycle.

21. The method of embodiment 16, wherein the cycling cells include at least some cells in the G1-phase of the cell cycle.

22. The method of embodiment 16, wherein the non-cycling cells include cells in the G0-phase of the cell cycle.

23. The method of embodiment 16, wherein the cycling cells include at least some cells in the G1-phase of the cell cycle and the non-cycling cells include at least some cells in the G0-phase of the cell cycle.

24. The method of embodiment 23,
wherein the first cellular analyte is identified in the cells in the G1-phase of the cell cycle, with no addition from the cells in the G0-phase of the cell cycle; or
wherein the first cellular analyte is identified in the cells in the G0-phase of the cell cycle, with no addition from the cells in the G1-phase of the cell cycle.

25. The method of embodiment 16, wherein the distinguishing includes identifying a second cellular analyte that is differentially expressed in cycling cells as compared to non-cycling cells.

26. The method of embodiment 25, wherein the second cellular analyte includes a cell cycle phase marker.

27. The method of embodiment 25, wherein the second cellular analyte includes Ki-67.

28. The method of embodiment 16, wherein the first cellular analyte is expressed in cells in the G1-phase.

29. The method of embodiment 28, wherein the first cellular analyte includes a DNA methyltransferase (DNMT).

30. The method of embodiment 28, wherein the first cellular analyte includes DNMT1.

31. The method of embodiment 16, wherein the distinguishing is performed using a single-cell method.

32. The method of embodiment 16, wherein the identifying is performed using a single-cell method.

33. The method of embodiment 16, wherein at least step (b) or (c) is performed using flow cytometry.

34. The method of embodiment 16, wherein step (b) is performed before step (c).

35. The method of embodiment 16, including:
d) identifying a level of the first cellular analyte.

36. A method for analyzing biological cells in a population of biological cells comprising cycling cells and non-cycling cells, including:
a) in the population of biological cells, distinguishing cells in G1 phase of the cell cycle from cells in G0 phase of the cell cycle; and
b) identifying a level of a first cellular analyte in the cells in the G1 phase, exclusive of a level of the first cellular analyte in the cells in the G0 phase; or
identifying a level of a first analyte in the cells in the G0 phase, exclusive of a level of the first analyte in the cells in the G1 phase.

37. The method of embodiment 36, wherein step (a) includes determining a second analyte in the cycling cells and non-cycling cells, where the second analyte is differentially expressed in cells in the G1 phase of the cell cycle as compared to cells in the G0 phase of the cell cycle.

38. The method of embodiment 36, including prior to step (a):
determining DNA content of the biological cells such that at least biological cells having a 2N DNA content are identified.

39. A method for analyzing a population of biological cells that includes cycling cells and non-cycling cells, comprising:
a) determining DNA content of individual biological cells in the population such that cells having a 2N DNA content are identified;
b) for cells having the 2N DNA content, distinguishing cells in G1 phase of the cell cycle from cells in G0 phase of the cell cycle; and
c) identifying a first cellular analyte in the cells in the G1 phase of the cell cycle, independent of the first cellular analyte in the cells in the G0 phase of the cell cycle; or
identifying a first cellular analyte in the cells in the G0 phase of the cell cycle, independent of the first cellular analyte in the cells in the G1 phase of the cell cycle.

40. The method of embodiment 39, wherein the population of biological cells includes eukaryotic cells.

41. The method of embodiment 39, wherein the population of biological cells includes mammalian cells.

42. The method of embodiment 39, wherein the population of biological cells includes cells from a human subject.

43. The method of embodiment 39, wherein the population of biological cells includes cells from blood, umbilical cord, spleen and/or bone marrow.

44. The method of embodiment 39, wherein the population of biological cells includes mononuclear cells.

45. The method of embodiment 39, wherein step (a) includes determining DNA content on a single-cell basis.

46. The method of embodiment 39, wherein step (a) includes contacting the biological cells with a substance that binds DNA and determining an amount of the substance bound by DNA in individual cells.

47. The method of embodiment 39, wherein step (a) includes staining the biological cells with a dye that binds DNA.

48. The method of embodiment 47, wherein the dye includes a fluorescent dye.

49. The method of embodiment 47, wherein the dye includes DAPI or propidium iodide.

50. The method of embodiment 39, wherein step (b) includes identifying a second cellular analyte that is differentially present in G1-phase cells as compared to G0-phase cells.

51. The method of embodiment 50, wherein identifying the second cellular analyte includes staining the cells with a reagent that binds to the second cellular analyte.

52. The method of embodiment 51, wherein the reagent includes an antibody.

53. The method of embodiment 51, wherein identifying the first cellular analyte includes staining the cells with a reagent that binds to the first cellular analyte.

54. The method of embodiment 53, wherein the reagent includes an antibody.

55. A method, comprising:
a) providing a population of cells;
b) fixing and/or permeabilizing the population of cells;
c) staining the population of cells for DNA content, a first analyte that is differentially expressed in cycling cells as compared to non-cycling cells, and a second analyte;
d) analyzing the stained cells;
e) distinguishing cycling cells from non-cycling cells, based on analysis of the first analyte in the stained cells; and
f) identifying the second analyte in the cycling cells exclusive of the non-cycling cells, or in the non-cycling cells exclusive of the cycling cells.

56. The method of embodiment 55, wherein the population of cells in (a) includes cycling cells or non-cycling cells.

57. The method of embodiment 55, wherein the population of cells in (a) includes cycling cells and non-cycling cells.

58. The method of embodiment 57, wherein the cycling cells include G1-phase cells and the non-cycling cells include G0-phase cells.

59. The method of embodiment 55, wherein the population of cells in (a) are eukaryotic cells.

60. The method of embodiment 55, wherein the population of cells in (a) are mammalian cells.

61. The method of embodiment 55, wherein the population of cells in (a) are human cells.

62. The method of embodiment 55, wherein the population of cells in (a) are mononuclear cells.

63. The method of embodiment 55, wherein the population of cells in (a) are from blood, umbilical cord, spleen and/or bone marrow.

64. The method of embodiment 55, wherein the population of cells in (a) has been treated with a candidate therapeutic agent.

65. The method of embodiment 64, wherein the candidate therapeutic agent affects levels of the second analyte.

66. The method of embodiment 64, wherein the candidate therapeutic agent includes azacitidine and/or decitabine.

67. The method of embodiment 55, wherein staining the population of cells for DNA content includes contacting the population of cells with a substance that binds to DNA.

68. The method of embodiment 67, wherein the substance that binds to DNA includes propidium iodide or DAPI.

69. The method of embodiment 55, wherein the first analyte is differentially expressed in G1-phase cells as compared to G0-phase cells.

70. The method of embodiment 55, wherein the first analyte includes a cell cycle phase marker.

71. The method of embodiment 55, wherein the second analyte is differentially present in G1-phase cells as compared to G0-phase cells.

72. The method of embodiment 55, wherein the second analyte includes a DNA methyltransferase (DNMT).

73. The method of embodiment 72, wherein the second analyte includes DNMT1.

74. The method of embodiment 55, wherein the analyzing in (d) includes determining DNA content, the first analyte, and the second analyte in the population of cells, based on the staining in (c).

75. The method of embodiment 55, wherein the analyzing in (d) includes single-cell methods.

76. The method of embodiment 55, wherein the analyzing in (d) includes flow cytometry, imaging or single-cell sequencing.

77. The method of embodiment 55, wherein in (e), the cycling cells include G1-phase cells and the non-cycling cells include G0-phase cells.

78. The method of embodiment 55, wherein a mean or median level of the second analyte is determined for cycling cells without addition from non-cycling cells.

79. A method, comprising:
a) providing a population of cells that includes cycling cells and non-cycling cells;
b) fixing and permeabilizing the population of cells.
c) staining the population of cells from (b) for DNA content, a first analyte that is differentially expressed in G1-phase cells as compared to G0-phase cells, and a second analyte;
d) analyzing the stained cells from (c) by flow cytometry;
e) distinguishing the G1-phase cells from the G0-phase cells, based on analysis of the first analyte in the stained population of cells; and
f) identifying the second analyte in the G1-phase cells exclusive of the G0-phase cells.

80. The method of embodiment 79, wherein the second analyte is differentially expressed in the G1-phase cells as compared to the G0-phase cells.

81. A method, comprising:
a) determining DNA content in individual cells in a population of biological cells containing cycling and non-cycling cells, to distinguish subpopulations of individual cells having a 2N DNA content, between a 2N and 4N DNA content, and a 4N DNA content;
b) within a subpopulation of cells having 2N, between 2N and 4N, and 4N DNA content, distinguishing an additional cell subpopulation based on differential presence of a first analyte in the additional subpopulation as compared to other cells in the subpopulation that are not in the additional subpopulation; and
c) within the additional subpopulation of cells, determining a second analyte.

82. The method of embodiment 81, wherein in step (b), the subpopulation of cells has a 2N DNA content and the additional cell subpopulation distinguished are cells in G1-phase of the cell cycle.

83. The method of embodiment 82, wherein in step (b), the first analyte is Ki-67.

84. The method of embodiment 82, wherein in step (c), the second analyte in DNMT1.

85. A method, comprising:
in a cell population that includes cycling cells and noncycling cells, distinguishing the cycling cells from the non-cycling cells; and
determining a cell cycle distribution of the cycling cells.

86. A method, comprising:
in a cell population that has been exposed to a candidate therapeutic agent, wherein the cell population of cells includes cycling cells and noncycling cells, distinguishing the cycling cells from the non-cycling cells; and
determining the cell cycle distribution of the cycling cells.

87. A method, comprising:
a) providing a population of cells that has been exposed to a candidate therapeutic agent, wherein the population of cells includes cycling cells and non-cycling cells;
b) distinguishing between the cycling cells and the non-cycling cells; and
c) identifying a first cellular analyte in the cycling cells, with no addition from the non-cycling cells.

88. The method of embodiment 87, wherein the distinguishing includes identifying a second cellular analyte that is differentially expressed in cycling cells as compared to non-cycling cells.

89. The method of embodiment 88, wherein the second cellular analyte is differentially expressed in G1-phase cells as compared to G0-phase cells.

90. The method of embodiment 89, wherein the second cellular analyte is expressed in G1-phase cells.

91. The method of embodiment 89, wherein the second cellular analyte is expressed in G1-phase cells and is not expressed in G0-phase cells.

92. The method of embodiment 88, wherein expression of the second cellular analyte is not affected by the candidate therapeutic agent.

93. The method of embodiment 88, wherein transcription of the second cellular analyte is not affected by the candidate therapeutic agent.

94. The method of embodiment 88, wherein the second cellular analyte includes analytes whose expression is activated by E2F.

95. The method of embodiment 88, wherein the second cellular analyte is selected from the group consisting of DNA polymerase, thymidine kinase, dihydrofolate reductase, cdc6, HsOrc1, and MCM5.

96. The method of embodiment 88, wherein the second cellular analyte includes Ki-67.

97. The method of embodiment 87, wherein the first cellular analyte includes analytes whose expression is activated by E2F.

98. The method of embodiment 87, wherein the first cellular analyte includes a DNA methyltransferase (DNMT).

99. The method of embodiment 98, wherein the DNMT includes DNA methyltransferase 1 (DNMT1).

100. The method of embodiment 87, wherein the candidate therapeutic includes a DNA methyltransferase inhibitor.

101. The method of embodiment 100, wherein the DNA methyltransferase inhibitor includes azacitidine and/or decitabine.

102. A method, comprising:
a) providing a population of cells that has been exposed to a candidate therapeutic agent;
b) fixing and/or permeabilizing the population of cells;
c) staining the population of cells for DNA content, for a first cellular analyte that is differentially expressed in cycling cells as compared to non-cycling cells, and for a second cellular analyte;
d) analyzing the stained cells;
e) distinguishing the cycling cells from the non-cycling cells, based on detection of the first analyte in the stained cells; and
f) detecting the second analyte in the cycling cells exclusive of the non-cycling cells, or in the non-cycling cells exclusive of the cycling cells.

103. The method of embodiment 102, wherein the population of cells in (a) includes cycling cells and non-cycling cells.

104. The method of embodiment 103, wherein the cycling cells include G1-phase cells and the non-cycling cells include G0-phase cells.

105. The method of embodiment 102, wherein the candidate therapeutic agent does not affect expression of the first analyte in G0- or G1-phase of the cell cycle.

106. The method of embodiment 102, wherein the candidate therapeutic includes a DNA methyltransferase inhibitor.

107. The method of embodiment 106, wherein the DNA methyltransferase inhibitor includes azacitidine and/or decitabine.

108. The method of embodiment 102, wherein the first cellular analyte includes analytes whose expression is activated by E2F.

109. The method of embodiment 102, wherein the first cellular analyte is differentially expressed in G1-phase cells as compared to G0-phase cells.

110. The method of embodiment 102, wherein the first cellular analyte is expressed in G1-phase cells.

111. The method of embodiment 102, wherein the first cellular analyte is expressed in G1-phase cells and is not expressed in G0-phase cells.

112. The method of embodiment 102, wherein expression of the first cellular analyte is not affected by the candidate therapeutic.

113. The method of embodiment 102, wherein the first cellular analyte is selected from the group consisting of DNA polymerase, thymidine kinase, dihydrofolate reductase, cdc6, HsOrc1, and MCM5.

114. The method of embodiment 102, wherein the first cellular analyte includes Ki-67.

115. The method of embodiment 102, wherein the second cellular analyte includes a DNA methyltransferase (DNMT).

116. The method of embodiment 115, wherein the DNMT includes DNA methyltransferase 1 (DNMT1).

117. The method of embodiment 102, wherein the analyzing in step (d) includes using a single-cell method.

118. The method of embodiment 117, wherein the single-cell method includes flow cytometry.

### EXAMPLES

The following examples are for illustrating various embodiments and are not to be construed as limitations.

### Example 1. Purpose of Study

These studies were designed to detect and/or measure an analyte in cycling cells that was present in a cell population containing cycling and non-cycling cells. The cycling cells of particular interest have a 2N/C DNA content. Healthy, normal cells have 2N DNA; tumor or cancer cells may have more than one stemline with different, but well defined 2C levels of DNA. The designation “2” refers to the genome complement that is a property of resting/quiescent (G0) or cycling cells in the G1 phase of the cell cycle. More specifically, the studies measured the single-cell levels of a gene product in G1 and S-phases of cells (i.e., cycling cells), in a population of cells containing G0 and cycling cells (i.e., in a population having both cycling and non-cycling subpopulations). The gene product was detected and measured, and levels of the gene product were expressed as center values (means/median) in single G1 or S-phase cells, without any contribution from the G0-phase cells.

In these studies, cells were assayed (measured) by flow cytometry and then analyzed by an invented method using a combination of commercially available software and proprietary code. In one of these studies presented here, the blood samples came from healthy donors on a phase I clinical trial. The cells having a 2N DNA amount (i.e., G1-phase cycling cells and G0-phase non-cycling cells) were detected by DNA content (i.e., using DNA staining with DAPI). S phase cells were identified by have >2N DNA<4N DNA. Blood samples were subjected to commercially available techniques for isolating the mononuclear fraction of cells (PBMC) that are severely depleted for red blood cells and granulocytes. Circulating T cells were identified by detection of two Type I analytes, CD3 and CD64. Cycling T cells were distinguished from G0-phase cells by detection of a Type II cellular analyte, Ki-67, that is exclusively expressed in cycling cells. G1 and S-phase cells as compared to G0-phase cells were distinguished by the combined quantified levels of DNA content and Ki-67. The target of the study, a Type III analyte (here, DNMT1) was measured in the cells, resulting in determination of DNMT1 levels in G1-phase cells and S phase cells without contamination by the background (non-specific light) from G0-phase cells. Because cycling T cells are rare, this method adequately measures DNMT1 in T cells. For example, conventional Western blots do not detect, or poorly/inconsistently detect DNMT1 in human blood samples. In this study, DNMT1 depletion was detected and quantified in 100% of subjects treated with a decitabine formulation. The levels of DNMT1 in a defined G1 phase was determined objectively as the reported values for the study. The higher level of DNMT1 in S phase relative to G1 phase was used as a sample quality assurance measure in untreated samples—i.e., the distinct cell cycle related expression of DNMT1 in T cells from untreated subjects was a reproducible, and expected index verifying that the sampling and preparation processes prior to fixation did not adversely affect the cells that were measured.

### Example 2. Antibodies and Reagents

CD3-FITC (human, clone REA613, catalog no. 130-113-138), CD64-PE-Vio770 (human, clone REA978, catalog no. 130-116-198), anti-Ki-67-PE (human and mouse, clone REA183, catalog no. 130-120-417), Tandem Signal Enhancer Reagent (TSE, catalog no. 130-099-888), and MACS Comp Bead Kit (anti-REA, catalog no. 130-104-693) were from Miltenyi Biotec Inc. Other reagents included Rabbit monoclonal anti-DNMT1 antibody [EPR3522] (catalog no. ab92314 from Abcam), F(ab′)2-Goat anti-Rabbit IgG (H+L) Cross-Adsorbed Secondary Antibody-Alexa Fluor 647 (catalog no. A-21246 from ThermoFisher Scientific/Invitrogen), Flow Cytometry Protein G Antibody Binding Beads (catalog no. 554 from Bangs Laboratories Inc), Luminex Viacount Reagent (catalog no. NCI 716261 from Fisher Scientific Company LLC), 4′,6-diamidino-2-phenylindole dihydrochloride (DAPI, catalog no. D8417-1MG from Millipore Sigma), bovine serum albumin (BSA, 30% solution, catalog no. A8327-50ML from Sigma Aldrich Inc), formaldehyde (16%, methanol free, ultra-pure, catalog no. 18814-10 from Polysciences Inc), methanol (HPLC grade, catalog no. A452SK4 from Fisher Scientific Company), dimethyl sulfoxide (DMSO, Hybri-Max grade, catalog no. D2650-100ML from Millipore Sigma), fetal bovine serum (FBS, catalog no. 25-514H from Genesee Scientific), and PBS (IOX, pH 7.4, catalog no. 70011044 from Life Technologies Corp).

### Example 3. Peripheral Blood Mononuclear Cell Isolation, Freezing and Storage

Blood was collected from individual human subjects, mononuclear cells were isolated, and frozen samples were prepared.

In detail, 8 ml human blood samples were collected in BD Vacutainer™ Glass Mononuclear Cell Preparation (CPT) Tubes [sodium heparin, catalog no. 14-959-51 D from Fisher Scientific (manufacturer no. BD 362753)], a one-step, closed-system tube for blood collection and mononuclear cell separation, and were processed according to the manufacturer's directions: invert 8 to 10 times to mix, centrifuge 1700 RCF for 20 minutes at room temperature, aspirate one-half of the plasma layer, then harvest and transfer the mononuclear layer to a 15 ml conical centrifuge tube. Mononuclear cells were washed twice with an excess (approximately 10 ml) of room temperature PBS and centrifuged (300 RCF for 10 to 15 minutes at room temperature). The cell pellet was resuspended in 1.5 ml 10% DMSO/90% FBS, equal aliquots were transferred to two 2 ml cryogenic vials, and the vials were frozen and stored (−80° C.) in a Corning CoolCell Freezing System for Cryogenic Vials [catalog no. EW-04392-00 from Cole-Parmer (manufacturer no. 432000)]. Samples were shipped on dry ice for next day delivery to the analyzing laboratory, where they were stored at −80° C. until processed, stained and assayed.

### Example 4. Preparation of Cells for Analysis

Mononuclear cells were thawed, counted, fixed, permeabilized, stained, and analyzed using flow cytometry, measuring forward and right-angle light scatters, Type I analytes (CD3; T cells) and CD64 (monocytes), Type II analytes Ki-67 and DNA, and Type Ill analyte DNMT1. The fluorescence labels can be variable, but were optimized in this case for the least fluorescence cross-talk or spillover, reducing the need for fluorescence compensation. In this case, the Type I and Type II analyte-reactive antibodies were labeled and therefore directly reporting probes. The Type III analyte used an unlabeled antibody and a secondary, labeled anti-antibody to produce the fluorescence signal proportional to the expression of the Type III analyte. In some cases, the Type III analytes will be detected with directly labeled antibodies or antibody-like detection reagents.

In detail, frozen samples of mononuclear cells were rapidly thawed in a 37° C. water bath for approximately 2 to 3 minutes and then placed immediately on ice. Live/dead cell counts were determined by dilution of an aliquot from each sample (1:20) with Luminex Viacount Reagent and then analysis on a Millipore (now Luminex) Guava Flow Cytometer, according to the manufacturer's protocol.

Mononuclear cells (<1 million cells per test) were resuspended in a final volume of 1 ml 2% BSA/PBS (in 12×75 mm polypropylene tubes) and fixed with 0.15% formaldehyde for 10 minutes at room temperature. Following this incubation and for the duration of the protocol, all tubes were maintained on ice, buffers were ice-cold, and centrifugations were performed at 350 RCF for 5 minutes at 4° C. After fixation, cells were washed once with 1 ml of PBS, centrifuged, and resuspended in 100 μl PBS. Cells were permeabilized by the dropwise addition of 900 μl (−80° C.) methanol (final concentration=90%), while simultaneously vortexing each tube, and incubation for 10 minutes. Cells were then stored in solution at −80° C.

In some cases, titrations of the formaldehyde fixing reagent may be used to optimize detection of Type III analytes.

In some cases, detergent reagents may be used to permeabilize cells to optimize detection of Type III analytes.

To prepare for staining, cell samples were brought to 4° C., then 1 ml PBS was added to each sample, and cells were centrifuged, aspirated, blocked with 1 ml 2% BSA/PBS for 30 minutes, and re-centrifuged. Staining was initiated by the addition of unlabeled anti-DNMT1 diluted antibody in a final volume of 100 μl of 2% BSA/PBS per test for 60 minutes. Cells were washed three times with 1 ml of 2% BSA/PBS for 10 minutes and centrifuged. After the third wash, CD3-FITC (2 μl/test), CD64-PE-Vio770 (2 μl/test), anti-Ki-67-PE (2 μl/test), diluted goat anti-rabbit-A647, and Miltenyi TSE (10 μ1/test) were added in a final volume of 100 μl of 2% BSA/PBS per test for 60 minutes. Cells were then washed three times with 1 ml 2% BSA/PBS for 10 minutes and centrifuged. After the third wash, samples were resuspended in 1 ml of PBS containing 0.5 μg/ml DAPI and transferred to a Nunc 96-well polypropylene deep well plate (2 ml, U-bottom, catalog no. 278743 from ThermoFisher Scientific).

All samples were measured on a dedicated CCSC Attune NxT Acoustic Focusing Flow Cytometer with Autosampler (ThermoFisher Scientific) at a flow rate of 200 μl per minute. Compensation was performed with MACS Compensation Beads (Miltenyi Biotec Inc.) and Protein G Antibody Binding Beads (Bangs Laboratories Inc.) according to the manufacturers' directions. Flow cytometry data was analyzed with WinList 3D (version 9.0.1, Verity Software House, Topsham, ME).

### Example 5. Properties of DNMT1 Cell Cycle-Related Expression

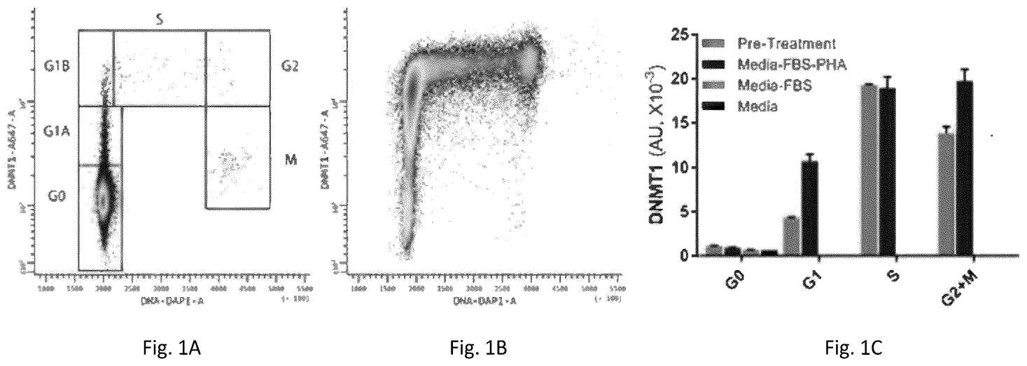

In the experiment presented in FIG. 1, PBMC were prepared from donor blood samples after incubation for 72 hours with or without the general T cell stimulant, phytohemaglutinin. The purpose of this treatment was to compare an overstimulated T cell population for cell cycle-related DNMT1 expression to the native expression in untreated cells. The cell cycle distribution was previously unknown for DNMT1 and T cells. The following paragraph describes the methods.

PBMCs were isolated from peripheral blood using BD Vacutainer CPT tubes (BD Biosciences, Ref. No. 362753), according to the manufacturer's instructions. Following isolation, cells were cultured in IMDM (Thermo Fisher Scientific, Catalog No. 21056023), IMDM/10% FBS, or IMDM/10% FBS/2.5 μg per ml eBioscience Phytohemagglutinin-L (PHA-L) (Thermo Fisher Scientific, Catalog No. 00-4977) for 72 hours at 37° C. in a humidified CO2 incubator. After 72-hours of incubation, PBMCs were fixed with 0.15% formaldehyde (Polysciences, Catalog No. 18814-10), permeabilized with 90% methanol (prechilled to −80° C.), and stored at −80° C. until assayed. To assay for DNMT1, cells were washed, blocked with PBS/2% FBS, and stained (for 1 hour at 4° C.) with Abcam Recombinant anti-DNMT1 Antibody [EPR3522] (0.0625 μg per test, Catalog No. ab92314), Goat anti-Rabbit IgG-Alexa Fluor 647 (0.0938 μg per test, Invitrogen, Catalog No. A21246), and relevant surface markers (CD45-FITC, CD3-PC7, CD14-PE). Cells were washed, stained with 0.5 μg per ml DAPI (Millipore Sigma, Catalog No. D8417), and analyzed with an Attune NxT Flow Cytometer (Thermo Fisher). FCS files were analyzed with WinList (Verity Software House) and Prism (GraphPad Software).

FIG. 1A shows the cell cycle phase distribution of DNMT1 in CD3+ T cells (i.e., T cells) in a sample of blood from a healthy donor, processed to a fixation and storage step soon after blood withdrawal. DNMT1 immunofluorescence is plotted on the Y-axis. The Y-axis is a Hyperlog plot (see Bagwell et al., Cytometry A, 2016; 89(12):1097-1105; and Bagwell et al., Cytometry A, 2005; 65(1):34-42) that plots values close to the origin in linear space and in log space as the values increase. DNA content is plotted on the X-axis. Note that the data show a large number of G0 cells (labeled G0) displaying background, non-specific fluorescence that is log-normally distributed. The G1 phase compartment is divided into “A” and “B” fractions, divided at the DNMT1 value that is minimally expressed in S phase. For purposes of this disclosure, this demonstrates an objective way to compartmentalize a continuous distribution in “early” (A) and “late” (B) fractions that may be useful. This is a known practice in the cell cycle cytometric literature that divides a relative continuum like G1 time by the expression relationship of two parameters. S and G2 phase designations are also conventional. The ratios of DNMT1 levels within each phase or sub-phase, with the exception of essentially the absence of expression in G0, were previously unknown. This is the pattern of expression in peripheral blood circulating T cells that have been processed to a stable fixed state soon after sampling. The same DNMT1 cell cycle expression pattern is elicited in rapidly proliferating T cells (FIG. 1B).

The same mononuclear cell sample was stimulated with PHA, processed to a stable fixed state at 72 hours, then stained and measured by flow cytometry as described. The expression DNMT1 expression patter of the CD3+ T cells is shown in FIG. 1B. In both FIGS. 1A and 1B, the same DNMT1 expression pattern is displayed with a maximum level of DNMT1 in G2 phase and variable, increasing expression in G1 with null expression in G0 (displaying background fluorescence). The variable G1 values represent cells that are in different cell cycle positions along a trajectory of time-related gene expression. Therefore, G1A cells are early, and G1B cells are late, and S phase cells are progressing through time as a function of DNA content (Jacobberger Systems Bio Encyclopedia Reference).

The plot in FIG. 1C shows the median fluorescence values on a linear scale in the main cell cycle phases, except for G2 which is combined with M, for T cells replicating at a rate set by natural in vivo circumstance (untreated) and T cells rapidly replicating after PHA treatment that abnormally over-stimulates. The levels of background in G0 cells and the level of DNMT1+background-related fluorescence in S phase cells are not significantly different, suggesting that like other cell cycle expressed genes, expression is tightly regulated. The significantly-different DNMT1+background related fluorescence values for G1 and G2+M cells are related to biology. The rapidly growing PHA stimulated cells express higher levels because the median cell is more advanced along a programmed cell cycle time related profile. The slower cell cycle times of the naturally stimulated T cells (untreated) are reflected by the lower values of cells that are at the median time point traversing G1. In G2+M, the differences reflect that active E2F remains active longer in over stimulated cells. The values for G1 fluorescence were calculated over the entire G1 (G1A+G1B).

In the experiment presented in FIGS. 1A-1C, staining included antibodies reactive with Type I and Type 111 analytes for purposes of this disclosure. To establish the cell cycle expression of DNMT1 in untreated T cells, it was not necessary to employ Type II analytes. Once established, best practice to determine the cell cycle expression of decitabine or azacitidine or other DNMT1-targeting lead compounds, candidate therapeutics, or approved therapeutics-treated cells incorporated a Type II analyte. The rationale is that the levels of DNMT1 in treated cells should/would decrease, and therefore, an independent method to establish cell cycle-related cell state detection was necessary.

It is evident, e.g., from but not exclusive to the data presented in FIG. 1 and Table 2, that DNMT1 can be a Type II analyte in diagnostic or prognostic assays, research studies, or clinical trials where lead compounds, candidate therapeutics, re-purposed therapeutics, or approved therapeutics do not target DNMT1. Thus, the categorization of analyte Type status is context dependent.

### Example 6. Reproducibility

The reproducibility of DNMT1 measurement was estimated on 4 independent preparations from the same donor sample. The relative DNMT1 values measured in either G1 or S phase T cells was less than 10% (Table 2, which is a value well within the range of values measured in other studies that are accepted as reproducible assays within the research and medical communities. This experiment also showed that any gain in information by including the markers CD4 and CD8 (not shown) was insignificant relative to the noise that spectral interference from additional fluorophores introduces to the DNMT1 channel (not shown). This does not mean that for any specific study in the future that these Type I analytes will not be employed. Therefore, median measurements of G1 and S phase values of DNMT1 are reproducible and equivalent in C3⁺ cells and CD3⁺/CD4⁺ cells. In Table 2, the means of median DNMT1 values are not very different and the reproducibility (CVs) are equivalent.

TABLE 2
DNMT1 in CD3⁺ and CD3⁺/CD4⁺ cells

| | |
|---|---|
| G1 DNMT1 | S DNMT1 |

CD3+

| | | |
|---|---|---|
| Mean | 1,187 | 14,026 |
| St dev | 76 | 706 |
| CV | 6% | 5% |

CD3+/CD4+

| | | |
|---|---|---|
| Mean | 1,185 | 16,376 |
| St dev | 54 | 1,054 |
| CV | 5% | 6% |

### Example 7. Data Analysis Approach

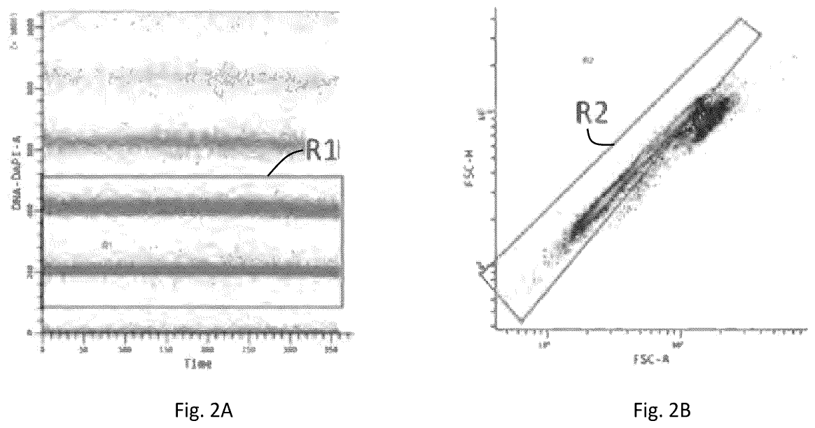

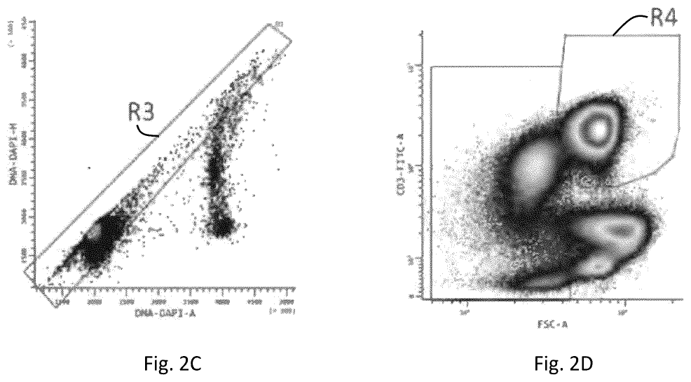

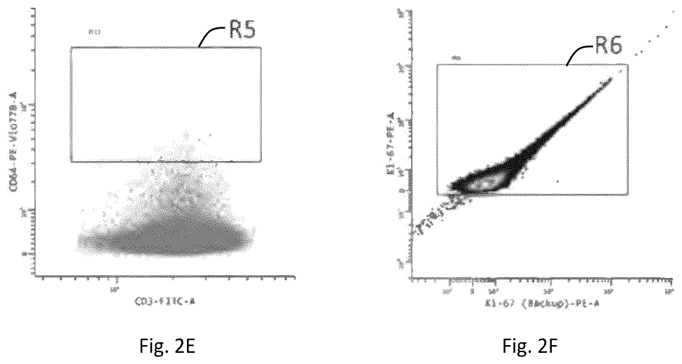

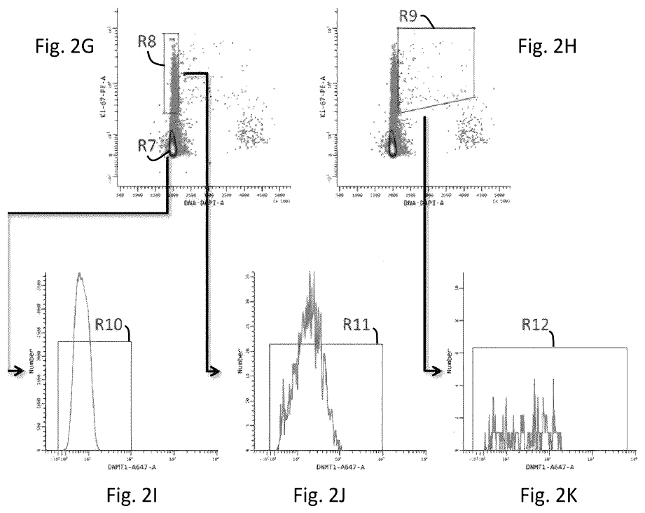

One scheme for reducing the multiparametric measurements to high quality DNMT1 values for G1 and S is illustrated in FIGS. 2A, 2B, 2C, 2D, 2E, 2F, 2G, 2H, 2I, 2J and 2K, and in the following text.

The raw data were assembled by the instrument as an event-row list in a standard (FCS) format. Each file contains meta-data specific to the sample measured. This file was read up by WinList (Verity Software House) and the analytical scheme depicted was used to select single, CD3 positive (CD3⁺)/CD64 negative (CD64⁻) cells that were alive at the time of fixation to a long-term stable state. From that point, three subpopulations were selected, G0, G1, and S, representing different states of cell cycle activity, and the median fluorescences were calculated. These values were automatically used to calculate the median DNMT1-specific immunofluorescence for G1 and S states by subtracting the linear median G0 background fluorescence (Fb) from the G1 or S DNMT1+background measurements (Ftot), which were then written down with metadata to a database file for transfer to Microsoft Excel or programs that read comma delimited values (CSV) files. Specifically, the calculation is DNMT1-specific fluorescence (Fs)=Ftot−Fb. In the following paragraphs and FIGS. 2A-2K, a detailed description of the post-measurement data processing is presented.

FIG. 2A, which initiated the analytical process, is a plot of DNA content versus time and illustrates that the sample run was initially checked for flow integrity with a plot of DNA content (Y-axis) versus time (X-axis). Any perturbations to the run were/would be identified as non-clustered data at this point and removed from the analysis by Boolean gating. FIG. 2A shows a run without perturbation. Region, R1, selects data that will be further processed.

FIG. 2B illustrates selection of all singlet events with DNA content from 2N→4N that were selected using R1 in FIG. 2A. FIG. 2B is a plot of the peak forward light scatter signals of all cells with 2N→4N DNA (Y-axis) versus the integrated light scatter signals of all cells with 2N→4N DNA (X-axis). This plot with the region, R2, is commonly referred to as “doublet discrimination”. In FIG. 2A, most of the aggregated and coincident events with more than two cells are eliminated from the analysis by the region/gate, R1. In FIG. 2B, clusters of events to the right of the region, R2, are doublets (two cells connected or entangled in fibers, largely produced by alcoholic fixation) and coincident events (two cells that were in the laser or light source beam at the same time. These events are eliminated in a Boolean fashion by creating a “gate”, g1 (satisfying the conditions of inclusion within the boundaries of R1 AND R2) and used for further processing.

FIG. 2C is a plot of the peak DAPI fluorescence signal of all singlet cells (Y-axis) versus the integrated DAPI fluorescence signals of all singlet cells (X-axis). The region R3 selects singlet G0, G1, S, G2, and partial M phase (prophase, prometaphase, and metaphase). The excluded events below the diagonal of R3 consist of late M phase cells (anaphase and telophase) and cell doublets that escaped the doublet discrimination of R2.

FIG. 2D illustrates selection of CD3+ T cells that were alive at the time of fixation. This is based on comparisons of independent live/dead assays correlated with cells of reduced light scatter (unlabeled region left of R4) in PBMC samples that have been subjected to storage in liquid nitrogen. The likely necrotic (lysed) T cells are the major population of reduced light scatter and dim or low CD3 expression (arrow). CD3+ T cells, alive at the point of fixation, were selected by created the region (R4 in FIG. 2D) and combining in a Boolean gate, g2 (g2=R1 AND R2 AND R3 AND R4) that are further processed and analyzed. The remaining clusters below the dead and live CD3+ T cells are other PBMC cell types (B cells, monocytes, etc.).

FIGS. 2E and 2F clean up the analyzed cell population further. FIG. 2E plots of CD64 immunofluorescence (a monocyte marker; Y-axis) versus CD3 (T lymphocytes; X-axis), showing only the CD3 positive region. The regions R5 selects any CD64 positive monocytes that also register as CD3 positive (possibly hetero-doublets that escaped doublet discrimination or monocytes with ingested or bound T cell fragments, etc.). FIG. 2F is a plot of Ki-67 (Y axis) versus Ki-67 measured at a different section of the PE fluorescence distribution (X axis). The rationale for this plot is that the very few, likely subcellular (e.g., bare nuclei) events that are supra-Ki-67 negative or supra-Ki-67 positive (e.g., replicating monocytes) are eliminated from the analyzed data set. This plot is not strictly necessary if those events are at a low frequency. The region, R6, defines these to be excluded events. Going forward analytically, a g3 gate is produced that is: g3=(R1 AND R2 AND R3 AND R4 AND NOT (R5 OR R6).

The previous data processing (FIGS. 1 and 2A-2F) use Type I and Type II analytes to produce an event data set that represents singlet CD3+ T cells in cell cycle biochemical states equivalent to the kinetically defined G0, G1, S, G2+early M cell cycle phases. At this point, restricted Type III analyte data are produced by as follows.

To start, Ki-67 is plotted versus DNA content to produce the cell cycle related Ki-67 expression pattern. Both FIGS. 2G and 2H are replicated plots of Ki-67 immunofluorescence (Y-axis) against DNA content (X-axis) to illustrate setting of regions R7, R8, and R9 that capture the most informative fractions of G0, G1, and S phase CD3+ T cells, used to measure background and phase-specific DNMT1 fluorescence plus background (Fb and Ftot, respectively). In this example, G0 is defined algorithmically by a central fraction (region R7, FIG. 2G) of the Ki-67/DNA defined G0 population. The rationale is that spurious events (e.g., Ki-67-negative, damaged S phase cells) are less likely to be included, and that for background measurements of an abundant population, the central values are most representative and robust. The G0 region, R7, is algorithmically a “contour” region, defined in WinList, and the contour level—i.e., the fraction of data capture around the center value—was 67% to 95% based on features of the baseline data (untreated) for each research subject, thus accounexample 1

ting for patient-to-patient variation in autofluorescence, antibody background binding background, and sample quality (fraction of damaged S phase cells, etc.).

The G1, R8, region (FIG. 2H) is set on “late” G1 cells. The formula for setting the bottom boundary of this region is based on the center and variance of the Ki-67 distribution in R7 (see FIG. 2L) and a multiplier (e.g., x times the background standard deviation). The variable statistical values, (center, variance) and the multiplier will change slightly replicate to replicate, more so from cell type to cell type, and significantly patient to patient or study to study, and reflects the sources of variation that this analytical approach takes into account. The R8 left and right boundaries depend on the center and variance of the DNA content (DAPI) distribution of the G0, R7, T cells. The top of the R8 region is defined by absence of Ki-67 positive events and is essentially open ended.

Setting the S phase region, R9, is based on the center and variance of the DNA content (DAPI) distribution in R7 to set the left-most boundary of R9 and an operationally determined multiplier to set the right-most boundary. The top boundary is set based on absence of Ki-67 positive events. The lower boundary is set based on the lower boundary of R7 and a Y direction multiplier that accounts for increasing Ki-67 in later (rightward) S phase cells. As is the case for R8, these R9 region setting variables will change slightly replicate to replicate, more so from cell type to cell type, and significantly patient to patient or study to study, and reflect the sources of variation that this analytical approach takes into account.

The rule-based objectively in the region-setting inherently facilitates reduction or complete removal of analyst arbitrary decisions, provides a system that is completely reproducible by outside, non-expert observers, and provides a system of record keeping that is important for good laboratory practice, good clinical laboratory practice, and clinical trial practice aimed at satisfying FDA, European, and world-wide therapeutic approvals.

The T cells that satisfy the Boolean logic (R1 AND R2 AND R3 AND R4 AND R7 AND NOT (R5 OR R6) were used to assemble the G0 DNMT1 channel fluorescence distribution data presented in (FIG. 2I). The distribution is broad. For the purpose of the study data presented in FIGS. 3-6, this fluorescence distribution was considered background and the median was calculated from region, R10. Since that time, we have evidence that this distribution is the sum of two underlying distributions. This could and likely represents G0 cells of distinctly different sizes (background is directly proportional to cell size), or there is a possibility that the more positive distribution represents cycling cells that have recently exited mitosis. Current practice is to fit the dimmer distribution to determine the background value, which provides a more uniform value patient to patient. However, either method is sufficient for good practice since the difference between the dim distribution and the total distribution is slight relative to values calculated for G1 and S phase cells. This is noted here because it is possible that in some studies—e.g., where a combination therapy affects the rate of mitotic exit or otherwise affects the existence of the cell states that comprise the two distributions, accounting for this feature may be informative.

The DNMT1 channel fluorescence distribution of G1-phase cells is shown in FIG. 2J. The combined G1 DNMT1-specific immunofluorescence plus background fluorescence distribution was examined in the untreated sample for abnormalities (e.g., two peaks). We expected a unimodal log-normal distribution based on experience. For the study presented in FIGS. 3-6, If abnormalities existed in the G1 distribution—i.e., an observable number of a dim population representing “early” G1 cells—the Ki-67 multiplier settings were increased. The more automated current practice, eliminates the necessity for this legacy feature. As with the G0 settings, this routine was only performed on the untreated, baseline sample, and for that research subject, subsequent samples were analyzed with untreated, baseline settings. This approach produced rule-based, semi-objective boundaries, and accounted for patient/subject unique characteristics of G1 combined DNMT1 immunofluorescence+background distributions. The completely inclusive region, R11, was used to calculated the median fluorescence of the combined distribution.

The DNMT1 specific and background combined distribution (FIG. 2K) for S phase cells defined by region (R9 in FIG. 2H) was calculated from the region, R12. The settings for S phase distribution determination and calculated were rule based, completely objective, but dependent on the settings defined for R8.

In control studies, using stable fluorescent microspheres, we determined that instrument variation (drift) was insignificant (not shown), but in each run (a set of samples measured sequentially over a contiguous period), a file representing measurements of stable, standardization microspheres was generated such that instrument performance throughout the fluorescence bands and intensities could be compared from run to run and adjusted mathematically to normalize the data to a standard instrument performance characteristic. Equally, instrument performance microspheres, supplied by the instrument manufacturer, were assayed with each run to establish that the instrument was performing as designed. Finally, staining standards (DNMT1 positive and dimly positive related cell lines were stained and measured) were assayed to monitor reproducibility of the staining technical process.

Statistics and Equations:

ANALYTE1=Median G1 DNMT1. This was calculated as Ftot−Fb=Fs, where Ftot=the Median G1 DNMT1-specific immunofluorescence+Background, Fb=Background, and Fs=ANALYTE1.

EVENTNUM1=Number of G1 cells in distribution R11.

ANALYTE2=Median S DNMT1. This was calculated as Ftot−Fb=Fs, where Ftot=the Median S DNMT1-specific immunofluorescence+Background, Fb=Background, and Fs=ANALYTE2.

EVENTNUM2=Number of G1 cells in distribution R12.

Ki-67 Levels: In the above-described analysis, note that in FIG. 2G, Ki-67 fluorescence channel values (Y-axis) are plotted against DNA content (X-axis). Based on DNA content, cells in both R7 and R8 contain a 2N amount of DNA, and therefore, are cycling, G1 phase cells (R8), or non-cycling, G0 phase cells (R7).

Cells in R8 are expressing Ki-67 (based on Ki-67 fluorescence shown on the Y-axis in FIG. 2G). Cells in R7 are not expressing Ki-67 or, at most, are expressing much lower levels of Ki-67 compared to the cells in R8. Because many published studies confirm that Ki-67 is expressed in cycling cells, the cells in R8 are conclusively cycling cells, and because the cells in R8 have 2N DNA content AND express Ki-67, they are in the G1 cell cycle phase. Because many published studies show that resting or quiescent cells do not express Ki67, cells in R7 are concluded to be non-cycling cells in the G0 cell cycle phase equivalent logic. For a sample of 8 healthy untreated human subjects, the ratio of G1 Ki-67-related fluorescence to the G0 Ki-67 channel fluorescence (background), determined in the same manner as presented above, is 24.7+/−7.1—i.e., the median late G1 cell produces a Ki67 immunofluorescence signal that is 25× background.

The S phase cells in R9 display high levels of Ki-67 related immunofluorescence. For a sample of 8 healthy untreated human subjects, the ratio of S phase Ki-67-related fluorescence to the G0 Ki-67 channel fluorescence (background) from the same data set as described in section 0154 is 54.6+/−21.3—i.e., the median S phase cell produces a Ki67 immunofluorescence signal that is 55× background.

In each sample discussed in sections 0154 and 0155, the median S phase Ki67-related immunofluorescence was higher than the median late G1 phase Ki-67-related immunofluorescence. Additionally, the G1 Ki-67 immunofluorescence to background and S phase Ki-67 immunofluorescence to background were calculated for these same subjects after treatment with decitabine, recovery, and retreatment. In each case, there were no significant differences between the mean ratios. When all the G1 Ki-67 background ratios were compared to all S Ki-67 to background ratios in a paired t test, the means were 23.7+/−6.3 versus 51.8+/−17.8, N=30, and the difference was highly significant (p<0.000001). This, together with uniformly high ratios of signal to background, strongly suggests that Ki-67 is a reproducible, sensitive, and stable cell cycle marker within the context of hypomethylating studies. Further, it can be strongly inferred that this cell cycle marker will satisfy requirements for a stable cell state analyte (Type II) in most studies where a Type II cell cycle analyte is needed.

Although the previously described signal to noise analysis demonstrates the robustness of Ki-67 as a Type II cell cycle analyte, it is possible that given enough data, any treatment that affects cell cycle progression (e.g., DNMT1 inhibitors at a high enough dose are S phase poisons) the possibility that the fraction of cells resident at different time points in G1 (i.e., early, mid, late phase) will demonstrate an effect on the median value of G1 Ki-67 is likely. However, the ratio between the Ki-67 signal in S phase and G1 phase will remain high, and therefore this ratio is a quality assurance feature of these methods.

DNMT1 Levels: FIGS. 2I and 2J are plots of DNMT1 levels (X-axis) against cell number (Y-axis). As indicated in the figures, FIG. 2I shows the DNMT1 channel fluorescence from cells in FIG. 2G that are enclosed within R7 boundaries (i.e., G0-phase cells) is low when compared to a similar plot (FIG. 2J) for cells in G1 (FIG. 2G, R8). Visually, it can be seen that the levels of DNMT1 are higher in the cells illustrated in FIG. 2J than in the cells illustrated in FIG. 2I. The conclusion is that G1-phase, cycling cells contain higher amounts of DNMT1 than G0-phase, non-cycling cells. Thus, confirming that this analysis produces the same result that was presented in FIG. 1. The difference between that more conventional analytical approach and this more rigorous analytical approach is rendering the methods objective with adjustable sensitivity (where the R8 lower boundary is placed, and readily programmable in an automated system.

From these data, one can intuitively see that the mean DNMT1 level for a population of cells comprising the R7+R8 subpopulations in FIG. 2G (G1+G0 cells) would be lower than the mean DNMT1 level for the R8 subpopulation of cells (G1 cells). These data show that, by separating 2N cells into their G0 and G1 subpopulations, using Ki-67 expression, the levels of DNMT1 in the G1 cells alone are more accurately determined, than if DNMT1 levels from the combination of G1- and G0-phase cells was used to represent G1-phase levels of DNMT1s

### Example 8. Expected DNMT1 Expression

In the cell cycle of normal cells, DNMT1 is expressed after activation of the positive-acting cell cycle E2F transcription factors, beginning in G1, and approaches a constant maximum that is reached in the first third of S phase (based on results presented in FIGS. 1, 2G, 2H, and 3). In all cases tested so far, this relationship is constant, however, the results presented here were check for a positive correlation between G1 and S phase DNMT1 baseline values, which was positive and significant. This suggests, that depending on level of stimuli and length of the cell cycle, that there is a general variable threshold at S phase entry; that a maximum is reached soon after entry, or alternatively, individuals may have different maxima set by the genetics and epi-genetics of their immune systems. Nevertheless, all of the expression features that we expected from experimental data were evident in 100% of the subjects in this study.

FIG. 3 indicates the uniformity of S phase levels of DNMT1 being higher than G1 levels of DNMT1 (P<0.000001; Paired t test, N=46), emphasizing the value of S phase levels as a quality assurance feature. On average, S phase values were 6× higher than the G1 levels. (range: 2.3-9.2). These data are from the clinical trial and were obtained by the methods described above (Light Scatters, CD3, CD64, Ki-67, DNA, DNMT1).

### Example 9. Effects of a DNMT1-Depleting Therapeutic (Decitabine) on DNMT1 Levels

Trial volunteer subjects were administered the DNMT1-depleting therapeutic, decitabine, at a low, non-cytotoxic dose. All patients were treated with the same dose and schedule. At various times after administration (i.e., 0-22 days), blood was collected from the subjects and mononuclear cells were isolated as described in Example 3. The mononuclear cells were then analyzed as described in Example 4.

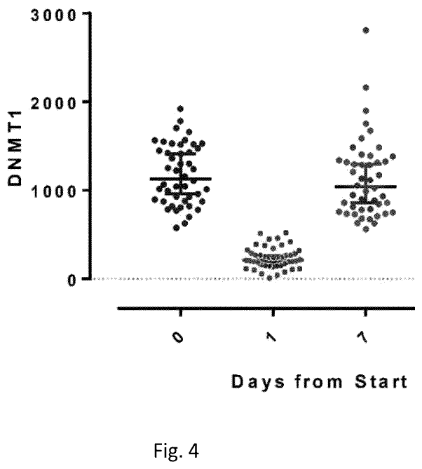

Plots of sample days from study start (X-axis) show that at 1 day after treatment with decitabine, all subjects demonstrated reduced levels of G1 DNMT1 (Y-axis)(FIG. 4). Thus, the assay sensitivity is 100% at this point.

These plots demonstrate the quantitative behavior of the system and the sensitivity (0-3,000 for G1 values, which can be extended to >10,000 if S phase is taken into account). At present, S phase values are useful for sample quality assurance (the assay works as expected). The numbers of cells obtained for S phase measurements are much less than G1 and therefore, the significant data to report consist of G1 values. It is also probable that G1, being the most variable part of the cell cycle in which cells can be alive and progressing without sufficient quantities of DNMT1, the activity of which will not be essential until S phase—suggesting that S phase values will be relatively constant because the cells in S are unaffected or least affected by drug treatment.

### Example 10. Effects of the DNMT1-Depleting Therapeutic on DNMT1 Levels in the Cell Cycle of Cycling Cells

To determine how using Ki-67 to distinguish G1-phase cells from G0-phase cells affects the determined levels of DNMT1 in G1-phase cells, compared to determining a DNMT1 value for all cells containing a 2N amount of DNA (G0+G1 cells), as may be done if Ki-67 were not used, the following study was performed.

The cells prepared and analyzed as in Example 9 (and shown in FIG. 4) were analyzed to determine the levels of DNMT1 in 2N cells that expressed Ki-67 (i.e., cycling, G1-phase cells) and these levels were compared to levels of DNMT1 in all cells (G0, G1, S and G2+M cells; this includes Ki-67-positive and Ki-67-negative cells). The results are shown in FIG. 5. These data and analysis are for one volunteer in the clinical trial presented in FIGS. 3 and 4.

In FIG. 5, the left-hand (blue) bar for each of days 0, 1, and 7 represents DNMT1 levels determined for G1-phase cells (excluding G0-phase cells) in mononuclear T cells prepared from this single research subject as described in Example 3. In FIG. 5, the right-hand (red) bar for each day represents DNMT1 levels for all cells in each sample (as if a western blot were performed on the PBMC preparation). The data show that the levels of DNMT1 in G1-phase cells displayed the significant differences shown in FIG. 4 (i.e., decreased DNMT1 due to decitabine, and subsequent recovery of DNMT1 levels at day 7). However, if DNMT1 levels were determined for all cells, there were no differences in DNMT1 levels between any of the time points. These data show that, by not using the information in Type I and Type II analytes—i.e., DNA and Ki-67 to distinguish G1-phase from G0-phase cells, much of the DNMT1 signal of G1 cells is displaced by the very large background contribution of other cells, including G0-phase cells.

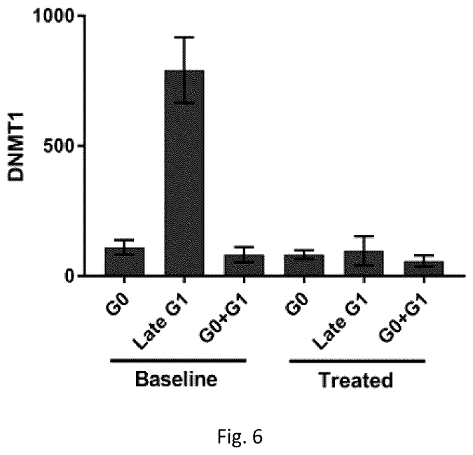

FIG. 6 shows data from another similar study that illustrates how distinguishing non-cycling G0 cells from cycling G1-phase cells provides better data for expression of some genes in the G1 phase, especially in cell populations containing cycling and non-cycling cells. FIG. 6 is a plot of DNMT1 levels (Y-axis) for cells from multiple untreated subjects (X-axis; indicated as “Baseline”) and for cells from subjects treated with the DNMT1-depleting therapeutic, decitabine (X-axis; indicated as “Treated”). On the X-axis, “G0” indicates cells with a 2N DNA content that did not express Ki-67. “Late G1” indicates cells with a 2N DNA content that expressed Ki-67. “G0+G1” indicates all cells with a 2N DNA content (i.e., cells with a 2N DNA content, independent of Ki-67 expression).

Looking at the DNMT1 values for the Baseline (untreated) cells in FIG. 6, it is seen that Late G1 cells contained 5-8 times the amount of DNMT1 as did G0+G1 cells. If Late G1 cells had not been distinguished from G0 cells using Ki-67 expression and, instead, the DNMT1 levels of G0+G1 cells had been used as a proxy for DNMT1 levels in G1-phase cells, actual expression of DNMT1 in G1-phase cells would have been obscured by lower expression levels of the larger number of G0 cells.

When the DNMT1 levels in Baseline (untreated), Late G1 cells in FIG. 6 is compared with DNMT1 levels in Treated, late G1 cells, it is seen that DNMT1 levels in the Treated cells were 5-8 times less than the levels in the Baseline cells. This difference represents the effect of decitabine on DNMT1 levels in cycling, G1-phase cells. This points to the value of the methods presented here that are not the obvious approach of the average practitioner in the field.

In contrast, if non-cycling, G0 cells had not been identified and excluded from G1-phase cells through Ki-67 detection, one would have compared DNMT1 levels in Baseline, G0+G1 cells with the levels in Treated, G0+G1 cells. In such an analysis, the conclusion would have been that decitabine had no effect on DNMT1 levels. This would have been an incorrect conclusion.

The benefits of excluding G0-phase cells from these types of analyses are clear.

We have done the Western blot equivalents of the above analysis and DNMT1 is not detectable by conventional Western blot methodologies.

1. A method for analyzing a population of biological cells that includes cycling cells and non-cycling cells, comprising:
a) determining DNA content of individual biological cells in the population such that cells having a 2N DNA content are identified;
b) for cells having the 2N DNA content, distinguishing cells in G1 phase of the cell cycle from cells in G0 phase of the cell cycle; and
c) identifying a first cellular analyte in the cells in the G1 phase of the cell cycle, independent of the first cellular analyte in the cells in the G0 phase of the cell cycle; or
identifying a first cellular analyte in the cells in the G0 phase of the cell cycle, independent of the first cellular analyte in the cells in the G1 phase of the cell cycle.

2. The method of claim 1, wherein the population of biological cells includes cells from blood, umbilical cord, spleen, effusions and/or bone marrow.

3. The method of claim 1, wherein the population of biological cells includes mononuclear cells.

4. The method of claim 1, wherein step (a) includes determining DNA content on a single-cell basis.

5. The method of claim 1, wherein step (b) includes identifying a second cellular analyte that is differentially present in G1-phase cells as compared to G0-phase cells.

6. The method of claim 5, wherein the second cellular analyte includes Ki-67.

7. The method of claim 1, wherein the first cellular analyte includes a DNA methyltransferase (DNMT1).

8. The method of claim 1, wherein the population of biological cells has been treated with a candidate therapeutic agent.

9. The method of claim 8, wherein the candidate therapeutic agent affects levels of the first cellular analyte and does not affect levels of the second cellular analyte.

10. The method of claim 8, wherein the candidate therapeutic agent includes azacytidine or decitabine.

11. A method, comprising:
a) providing a population of cells that has been exposed to a candidate therapeutic agent;
b) fixing and/or permeabilizing the population of cells;
c) staining the population of cells for DNA content, for a first cellular analyte that is differentially expressed in cycling cells as compared to non-cycling cells, and for a second cellular analyte;
d) analyzing the stained cells;
e) distinguishing the cycling cells from the non-cycling cells, based on detection of the first analyte in the stained cells; and
f) detecting the second analyte in the cycling cells exclusive of the non-cycling cells, or in the non-cycling cells exclusive of the cycling cells.

12. The method of claim 11, wherein the population of cells in (a) includes cycling cells and non-cycling cells.

13. The method of claim 11, wherein the candidate therapeutic includes a DNA methyltransferase inhibitor.

14. The method of claim 11, wherein the first cellular analyte is differentially expressed in G1-phase cells as compared to G0-phase cells.

15. The method of claim 11, wherein expression of the first cellular analyte is not affected by the candidate therapeutic.

16. The method of claim 11, wherein the first cellular analyte is selected from the group consisting of Ki-67, DNA polymerase, thymidine kinase, dihydrofolate reductase, cdc6, HsOrc1, and MCM5.

17. The method of claim 11, wherein the second cellular analyte includes a DNA methyltransferase (DNMT).

18. The method of claim 11, wherein the analyzing in step (d) includes using a single-cell method.
